(19) United States
(12) Patent Application Publication
(10) Pub. No.: US 2025/0370170 A1
(43) Pub. Date: Dec. 4, 2025

(54) TUNABLE LENS WITH FLUID-FILLED CHAMBERS COMPRISING DIFFERENT MATERIALS

(71) Applicant: Apple Inc., Cupertino, CA (US)

(72) Inventors: Igor Stamenov, Manteca, CA (US); Ferdinand S. Melkonyan, Longmont, CO (US); Fredric R. Askham, Westminster, CO (US); James E. Pedder, Oxon (GB); Thomas J. Aldrich, Boulder, CO (US)

(21) Appl. No.: 19/224,291

(22) Filed: May 30, 2025

Related U.S. Application Data
(60) Provisional application No. 63/654,264, filed on May 31, 2024.

(51) Int. Cl. G02B 3/12; C07C 43/225; C08G 65/26; C08G 75/08; C08G 77/00; C08G 77/24

(52) U.S. Cl. CPC G02B 3/12; C07C 43/225; C08G 65/2609; C08G 65/2639; C08G 75/08; C08G 77/24; C08G 77/80

(57) ABSTRACT

Materials and devices are provided. A lens module may include a first optical fluid having a density at 20° C. ranging from 1.20 to 1.85 g/mL and a refractive index of the ranging from 1.30 to 1.55 and a second optical fluid having a density at 20° C. ranging from 0.70 to 1.30 g/mL and a refractive index of the ranging from 1.50 to 1.75. The first and second optical fluids may be substantially free of any per- and polyfluoroalkyl substances (PFAS).

### CROSS-REFERENCE TO RELATED APPLICATIONS

This application claims the benefit of U.S. Provisional Application No. 63/654,264, “Tunable Lens with Fluid-Filled Chambers Comprising Different Materials,” filed May 31, 2024, which is incorporated by reference herein in its entirety for all purposes.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

Electronic devices may include displays and other components for presenting content to users. The electronic devices may be wearable electronic devices. A wearable electronic device such as a head-mounted device may include displays formed from one or more display panels for displaying visual content to a user. In some cases, gravity may induce optical aberrations (e.g., coma, astigmatism, spherical gradient) in fluid-filled adjustable lenses. Membrane sagging caused by vertical hydrostatic pressure variation controlled by gravity may generate optical aberrations and/or distortions in fluid-filled adjustable lenses impacting display of visual content by the lens system of the head-mounted device. As such, lenses of the lens module may include a first fluid-filled chamber and a second fluid-filled chamber selected such that the fluid-filled chambers mutually compensate one another for gravity induced aberrations/membrane sagging. Previously available fluids used for gravity sag compensation may include per- and polyfluoroalkyl substances (PFAS). PFAS includes a group of synthetic fluorine containing chemical compounds used in a multitude of consumer products. A subset of PFAS compounds are considered as persistent organic pollutants.

Accordingly, the present disclosure is directed towards fluids (e.g., substantially PFAS-free fluids with less than 50 parts per million (ppm) PFAS) for use in lens modules of electronic devices. In the following discussion, a lens module of an electronic device may include two fluid-filled chambers with optical fluids having different properties to mitigate gravity induced aberrations/membrane sagging. The optical fluids may be formed from PFAS-free materials. A first optical fluid may have a high density and low refractive index whereas a second optical fluid may have a low density and high refractive index. For example, in certain embodiments, the first optical fluid may have a refractive index that ranges from about 1.30 to 1.55 and a density that ranges from about 1.20 to 1.85 g/mL. The second optical fluid may have a refractive index that ranges from about 1.50 to 1.75 and a density that ranges from about 0.70 to 1.30 g/mL.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings described below in which like numerals refer to like parts.

FIG. 1 is a block diagram of an electronic device, according to embodiments of the present disclosure;

FIG. 2 is a schematic diagram of a top view of the electronic device of FIG. 1 in an illustrative configuration in which the electronic device is a head-mounted device, according to embodiments of the present disclosure;

FIG. 3 is a schematic diagram of a cross-sectional side view of a lens module of FIG. 2 including one or more lens elements, according to embodiments of the present disclosure;

FIG. 4 is a cross-sectional side view of a lens module including a fluid-filled lens, according to embodiments of the present disclosure;

FIG. 5 is a schematic embodiment of a cross-sectional side view of the lens module and the fluid-filled lens of FIG. 4 including an illustrative adjustment of a shape of a first lens element, according to embodiments of the present disclosure;

FIG. 6A is a schematic diagram of a cross-sectional side view of the lens module and the fluid-filled lens of FIG. 4 including a lens element formed by an electroactive polymer (EAP), according to embodiments of the present disclosure;

FIG. 6B is a schematic diagram of the EAP lens element of FIG. 6A, according to embodiments of the present disclosure;

FIG. 6C is a schematic diagram of the EAP lens element of FIG. 6A including one or more fingers patterned into an electrode, according to embodiments of the present disclosure; and

FIG. 7 is a schematic embodiment of a cross-sectional side view of a lens module including a first fluid-filled chamber and a second fluid-filled chamber, according to embodiments of the present disclosure.

### DETAILED DESCRIPTION OF SPEClFIC EMBODIMENTS

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. All numerical values within the detailed description herein are modified by “about” the indicated value, and take into account experimental error and variations that would be expected by a person having ordinary skill in the art. For example, “about” or “approximately” may refer to ±0.2%, ±0.5%, ±1%, ±2, ±5%, ±10%, or ±15%.

For purposes herein, a “polymer” has two or more of the same or different monomer (“mer”) units. “Different” in reference to mer units indicates that the mer units differ from each other by at least one atom or are different isomerically. Unless otherwise indicated, the terms “substituted” and “modified” means that at least one hydrogen atom has been replaced with at least one non-hydrogen group, such as a hydrocarbyl group, a heteroatom or heteroatom-containing group, such as halogen (such as Br, Cl, F or I) or at least one functional group such as—NR*₂, —OR*, —SeR*, —TeR*, —PR*₂, —AsR*₂, —SbR*₂, —SR*, —BR*₂, —SiR*₃, —GeR*₃, —SnR*₃, —PbR*₃, —(CH₂)_{q}—SiR*₃, and the like, where q is 1 to 10 and each R* is independently hydrogen, a hydrocarbyl or halocarbyl, and two or more R* may join together to form a substituted or unsubstituted completely saturated, partially unsaturated, or aromatic cyclic or polycyclic ring structure, or where at least one heteroatom has been inserted within a hydrocarbyl ring.

In the description herein, per- and polyfluoroalkyl substances (PFAS) are defined as: any substance that contains at least one fully fluorinated methyl (CF₃) or methylene (CF₂) carbon atom (without any H/Cl/Br/I attached to it). A substance that only contains the following structural elements is also excluded from the scope of the PFAS definition: CF₃—X or X—CF₂—X′ where X═—OR or —NRR′ and X′=methyl (—CH₃), methylene (—CH₂—), an aromatic group, a carbonyl group (—C(O)—), —OR″, —SR′, or —NR″R′″, and where R/R′/R″/R″′ is a hydrogen (—H), methyl (—CH₃), methylene (—CH₂—) an aromatic group, or a carbonyl group (—C(O)—). In the description herein, “PFAS-free fluids” are defined as fluids with less than 50 ppm PFAS substances.

All ranges expressed herein should include both end points as two specific embodiments unless specified or indicated to the contrary.

Electronic devices may include displays and other components for presenting content to users. The electronic devices may be wearable electronic devices. A wearable electronic device such as a head-mounted device may include displays formed from one or more display panels for displaying visual content to a user. A lens system may be used to allow the user to focus on the display and view the visual content. The lens system may include one or more lens modules. For example, the lens system may include a left lens module that is aligned with a user's left eye and a right lens module that is aligned with a user's right eye. The lens modules may include lenses that are adjustable. For example, fluid-filled adjustable lenses may be used to adjust the lens modules to display content to users with differing personal vision.

In some cases, gravity may induce optical aberrations (e.g., coma, astigmatism, spherical gradient) in fluid-filled adjustable lenses. Gravity sag may generate optical aberrations and/or distortions in fluid-filled adjustable lenses impacting display of visual content by the lens system of the head-mounted device. As such, lenses of the lens module may include a first fluid-filled chamber and a second fluid-filled chamber to correct and/or compensate for gravity induced aberrations in fluid-filled adjustable lenses. In certain embodiments, fluids of the first fluid-filled chamber and the second fluid-filled chamber may be selected such that the fluid-filled chambers mutually compensate one another for gravity induced aberrations. The fluids may have different refractive indices and densities.

Previously available fluids used for gravity sag compensation may include per- and polyfluoroalkyl substances (PFAS). PFAS includes a group of synthetic fluorine containing chemical compounds used in a multitude of consumer products. PFAS are long-lasting in the environment due to the persistence of some carbon-fluorine bonds. A subset of PFAS compounds are considered as persistent organic pollutants.

Accordingly, the present disclosure is directed towards substantially PFAS-free fluids for use in lens modules of electronic devices. In some embodiments, the PFAS-free fluids of this disclosure may be used in combination with fluids containing PFAS materials. In the following discussion, a lens module of an electronic device may include two fluid-filled chambers with optical fluids having different properties to mitigate gravity effects. The optical fluids may be formed from PFAS-free materials. A first optical fluid may have a high density and low refractive index whereas a second optical fluid may have a low density and high refractive index. It should be noted that materials with C—F bonds and/or S—F bonds may demonstrate low refractive index and high density that may be desirable for use in the first optical fluid of the lens module. Accordingly, fluorinated, PFAS-free functional groups may be used as building blocks for PFAS-free fluids for adjustable fluid-filled lenses.

In certain embodiments, the first optical fluid may have a refractive index that is less than about 1.55 (e.g., range from about 1.30 to 1.55) and a density that is greater than about 1.20 g/cm³ (e.g., range from about 1.20 to 1.85 g/mL). The first optical fluid having a high density and low refractive index may include a modified polyether material, a modified polysiloxane material, a modified polyester material, and the like. The second optical fluid may have a refractive index that is greater than 1.50 (e.g., range from about 1.50 to 1.75) and a density that is less than 1.25 g/cm³ (e.g., range from about 0.70 to 1.30 g/mL). The second optical fluid having a low density and a high refractive index may include a modified polysulfide material, a modified polypropylene material, and the like. The difference in refractive index between the first optical fluid and the second optical fluid may be about 0.16, 0.17, 0.20, 0.21, 0.22, 0.27, 0.29, 0.30, or 0.31, the difference may range between about 0.16 and 0.31, about 0.16 and 0.30, about 0.22 to about 0.30, at least 0.16, and the like. The difference in density between the first optical fluid and the second optical fluid may be about 0.32 g/cm³, 0.35 g/cm³, 0.44 g/cm³, 0.45 g/cm³, 0.47 g/cm³, 0.48 g/cm³, 0.56 g/cm³, 0.57 g/cm³, 0.61 g/cm³, 0.68 g/cm³, 0.70 g/cm³, 0.74 g/cm³, 0.81 g/cm³, 0.85 g/cm³, 0.91 g/cm³, 0.96 g/cm³, between about 0.32 g/cm³ and 0.96 g/cm³, between about 0.56 g/cm³ and 0.96 g/cm³ at least 0.32 g/cm³, at least 0.56 g/cm³, and the like.

In some embodiments, the first optical fluid with the low refractive index and the high density may include a polyether material modified with one or more functional groups. The functional groups may include one or more trifluoromethoxy methyl groups, difluoromethyl groups, pentafluorosulfanyl methyl groups, pentafluorophenyl groups, (2-ethoxyethyl) pentafluoro sulfane groups, a pentafluorosulfanyl group, and/or a pentafluorosulfanyl methyl group. The functionalized polyether material may have a density at 20° C. ranging about 1.45 to 1.59 g/mL. The functionalized polyether material may have a refractive index ranging from about 1.35 to 1.47 The second optical fluid with the low density and high refractive index may include a polysulfide material or a polydiphenylsulfide material. The polysulfide material and/or the polydiphenylsulfide material may have a density at 20° C. ranging about 0.95 to 1.15 g/mL. The polysulfide material and/or the polydiphenylsulfide material have a refractive index ranging from about 1.51 to 1.59. The difference in refractive index between the first and second optical fluid may range from about 0.04 to 0.24. The difference in density between the first and second optical fluid may range from about 0.30 to 0.65.

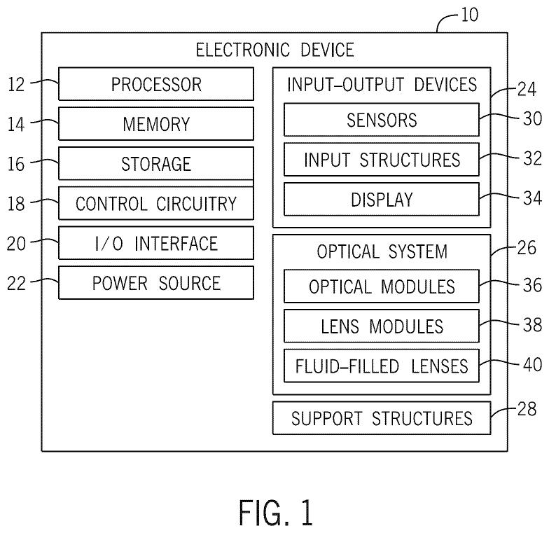

FIG. 1 is a block diagram of an electronic device 10, according to embodiments of the present disclosure. The electronic device 10 may include, among other things, one or more processors 12 (collectively referred to herein as a single processor for convenience, which may be implemented in any suitable form of processing circuitry), memory 14, nonvolatile storage 16, control circuitry 18, an input/output (I/O) interface 20, a power source 22, one or more input/output devices 24, an optical system 26, one or more support structures 28, one or more additional component, or a combination thereof. The various functional blocks shown in FIG. 1 may include hardware elements (including circuitry), software elements (including machine-executable instructions) or a combination of both hardware and software elements (which may be referred to as logic). The processor 12, memory 14, the nonvolatile storage 16, the control circuitry 18, the input/output (I/O) interface 20, the power source 22, the input/output devices 24, and the optical system 26, may each be communicatively coupled directly or indirectly (e.g., through or via another component, a communication bus, a network) to one another to transmit and/or receive signals between one another. It should be noted that FIG. 1 is merely one example of a particular implementation and is intended to illustrate the types of components that may be present in the electronic device 10.

By way of example, the electronic device 10 may include any suitable device, including computers, cellular telephones, wearable electronic devices, head-mounted devices, smart glasses, wristwatch devices, and other similar electronic devices. It should be noted that the processor 12 and other related items in FIG. 1 may be embodied wholly or in part as software, hardware, or both. Furthermore, the processor 12 and other related items in FIG. 1 may be a single contained processing module or may be incorporated wholly or partially within any of the other elements within the electronic device 10. The processor 12 may be implemented with any combination of general-purpose microprocessors, microcontrollers, digital signal processors (DSPs), field programmable gate array (FPGAs), programmable logic devices (PLDs), controllers, state machines, gated logic, discrete hardware components, dedicated hardware finite state machines, or any other suitable entities that may perform calculations or other manipulations of information. The processors 12 may include one or more application processors, one or more baseband processors, or both, and perform the various functions described herein.

In the electronic device 10 of FIG. 1, the processor 12 may be operably coupled with a memory 14 and a nonvolatile storage 16 to perform various algorithms. Such programs or instructions executed by the processor 12 may be stored in any suitable article of manufacture that includes one or more tangible, computer-readable media. The tangible, computer-readable media may include the memory 14 and/or the nonvolatile storage 16, individually or collectively, to store the instructions or routines. The memory 14 and the nonvolatile storage 16 may include any suitable articles of manufacture for storing data and executable instructions, such as random-access memory, read-only memory, rewritable flash memory, hard drives, and optical discs. In addition, programs (e.g., an operating system) encoded on such a computer program product may also include instructions that may be executed by the processor 12 to enable the electronic device 10 to provide various functionalities.

The control circuitry 18 of the electronic device 10 may include storage and processing circuitry for controlling the operation of the electronic device 10. The control circuitry 18 may implement control operations for device 10 (e.g., data gathering operations, operations involved in processing three-dimensional facial image data, operations involving the adjustment of components using control signals, etc.). The control circuitry 18 may include wired and wireless communications circuitry. For example, control circuitry 18 may include radio-frequency transceiver circuitry such as cellular telephone transceiver circuitry, wireless local area network (WI-FI) transceiver circuitry, millimeter wave transceiver circuitry, and/or other wireless communications circuitry. During operation, the communications circuitry of the control circuitry 18 of the electronic device 10 may be used to support communication between one or more additional electronic devices. For example, one electronic device may transmit video and/or audio data to another electronic device. The communications circuitry may be used to allow data to be received by device 10 from external equipment (e.g., a tethered computer, a portable device such as a handheld device or laptop computer, online computing equipment such as a remote server or other remote computing equipment, or other electrical equipment) and/or to provide data to external equipment.

The I/O interface 20 may enable electronic device 10 to interface with various other electronic devices. In some embodiments, the I/O interface 20 may include an I/O port for a hardwired connection for charging and/or content manipulation using a standard connector and protocol, such as the Lightning connector, a universal serial bus (USB), or other similar connector and protocol. The network interface 20 may include, for example, one or more interfaces for a personal area network (PAN), such as an ultra-wideband (UWB) or a BLUETOOTH network, a local area network (LAN) or wireless local area network (WLAN), such as a network employing one of the IEEE 802.11x family of protocols (e.g., WI-FI), and/or a wide area network (WAN), such as any standards related to the Third Generation Partnership Project (3GPP), including, for example, a 3^{rd} generation (3G) cellular network, universal mobile telecommunication system (UMTS), 4^{th} generation (4G) cellular network, Long Term Evolution (LTE) cellular network, Long Term Evolution License Assisted Access (LTE-LAA) cellular network, 5th generation (5G) cellular network, and/or New Radio (NR) cellular network, a 6th generation (6G) or greater than 6G cellular network, a satellite network, a non-terrestrial network, and so on. In particular, the network interface 20 may include, for example, one or more interfaces for using a cellular communication standard of the 5G specifications that include the millimeter wave (mmWave) frequency range (e.g., 24.25-300 gigahertz (GHz)) that defines and/or enables frequency ranges used for wireless communication. The power source 22 of the electronic device 10 may include any suitable source of power, such as a rechargeable lithium polymer (Li-poly) battery and/or an alternating current (AC) power converter.

The input-output devices 24 of the electronic device may include one or more sensors 30, one or more input structures 32, one or more displays 34, one or more additional components, or a combination thereof. The input-output devices 24 may be used to allow a user to provide device 10 with user input. Input-output devices 24 may also be used to gather information on the environment in which device 10 is operating. Output components of the input-output devices 24 may allow the electronic device 10 to provide a user with output and may be used to communicate with external electrical equipment.

The sensors 30 of the input-output devices 24 may include, for example, three-dimensional sensors (e.g., three-dimensional image sensors such as structured light sensors that emit beams of light and that use two-dimensional digital image sensors to gather image data for three-dimensional images from light spots that are produced when a target is illuminated by the beams of light, binocular three-dimensional image sensors that gather three-dimensional images using two or more cameras in a binocular imaging arrangement, three-dimensional lidar (light detection and ranging) sensors, three-dimensional radio-frequency sensors, or other sensors that gather three-dimensional image data), cameras (e.g., infrared and/or visible digital image sensors), gaze tracking sensors (e.g., a gaze tracking system based on an image sensor and, if desired, a light source that emits one or more beams of light that are tracked using the image sensor after reflecting from a user's eyes), touch sensors, buttons, force sensors, sensors such as contact sensors based on switches, gas sensors, pressure sensors, moisture sensors, magnetic sensors, audio sensors (microphones), ambient light sensors, microphones for gathering voice commands and other audio input, sensors that may gather information on motion, position, and/or orientation (e.g., accelerometers, gyroscopes, compasses, and/or inertial measurement units that include all of these sensors or a subset of one or two of these sensors), fingerprint sensors and other biometric sensors, optical position sensors (optical encoders), and/or other position sensors such as linear position sensors, and/or other sensors. The sensors 30 may include proximity sensors (e.g., capacitive proximity sensors, light-based (optical) proximity sensors, ultrasonic proximity sensors, and/or other proximity sensors). Proximity sensors may, for example, be used to sense relative positions between a user's nose and lens modules in the electronic device 10.

The input structures 32 of the input-output devices 24 may enable a user to interact with the electronic device 10. The input structures 32 may include haptic output devices (e.g., vibrating components), light-emitting diodes and other light sources, speakers such as ear speakers for producing audio output, and other electrical components. For example, the user may interact with the electronic device 10 by pressing a button to increase or decrease a volume level, pressing a button to modify displayed content, and the like. The display 34 of the input-output devices 24 may include a display to display images for a user of a head-mounted device (e.g., example of the device 10). The display 34 may include organic light-emitting diode displays or other displays based on arrays of light-emitting diodes, liquid crystal displays, liquid-crystal-on-silicon displays, projectors or displays based on projecting light beams on a surface directly or indirectly through specialized optics (e.g., digital micromirror devices), electrophoretic displays, plasma displays, electrowetting displays, or any other suitable displays.

In some embodiments, a user may observe physical objects through the display 34 while computer-generated content is overlaid on top of the physical objects by presenting computer-generated images on the display 34. The display 34 may be a transparent or translucent display formed from a transparent or translucent pixel array (e.g., a transparent organic light-emitting diode display panel) or may be formed by a display device that provides images to a user through a transparent structure such as a beam splitter, holographic coupler, or other optical coupler (e.g., a display device such as a liquid crystal on silicon display). Additionally and/or alternatively, the display 34 may be an opaque display that blocks light from physical objects when a user operates the head-mounted device. In this type of arrangement, a pass-through camera may be used to display physical objects to the user. The pass-through camera may capture images of the physical environment and the physical environment images may be displayed on the display 34 for viewing by the user. Additional computer-generated content (e.g., text, game-content, other visual content, etc.) may optionally be overlaid over the physical environment images to provide an extended reality environment for the user. In embodiments, in which the display 34 is opaque, the display 34 may also optionally display entirely computer-generated content (e.g., without displaying images of the physical environment).

In some embodiments, the display 34 of the electronic device 10 may operate in combination with the optical system 26. For example, a single display may produce images for both eyes or a pair of displays (e.g., display modules, display assemblies, stereoscopic displays) may be used to display images. In some embodiments, the focal length and positions of one or more lenses of multiple displays (e.g., left and right eye displays), may be selected so that any gap present between the displays will not be visible to a user (e.g., so that the images of the left and right displays overlap or merge seamlessly). The displays may present two-dimensional content (e.g., a user notification with text), three-dimensional content (e.g., a simulation of a physical object such as a cube), or a combination thereof.

The optical system 26 may include one or more optical modules 36, one or more lens modules 38, one or more fluid-filled lens 40, a positioner 42, one or more additional components (e.g., partially reflective mirrors that reflect 50% of incident light, linear polarizers, quarter wave plates, reflective polarizers, circular polarizers, reflective circular polarizers, etc.), or a combination thereof. The optical module 36 may include a first optical module corresponding to a user's right eye and a second optical module corresponding to the user's left eye. The optical module 36 may also include a positioner to modify a position of one or more components of the optical system 26. The lens modules 38 may include one or more lens elements, one or more lens housings, one or more lens actuators, one or more additional components, or a combination thereof. The lens elements may be rigid, elastomeric, or semi-rigid and may have any desired shape. The lens elements in the lens module may form the fluid-filled lenses. The fluid-filled lenses 40 may include one or more fluid-filled chambers (e.g., two or more fluid-filled chambers) that include optical fluid interposed between a first lens element and a second lens element. The fluid-filled chambers may be filled with optical fluid. The optical fluid may be a liquid, gel, or gas with a pre-determined index of refraction. The optical fluid may sometimes be referred to as an index-matching oil, an optical oil, an optical fluid, an index-matching material, an index-matching liquid, etc. The first lens element and the second lens element may have the same index of refraction or may have different indices of refraction. The optical fluids that fill the chambers may have different respective refractive indices and densities to compensate for gravity induced aberrations, as a thickness of the chambers may be affected due to a bulge caused by gravity induced hydrostatic pressure variation of the fluid column.

The electronic device 10 may include the support structures 28 (e.g., housing walls, straps, etc.). In some instances, the electronic device 10 is a head-mounted device (e.g., a pair of glasses, goggles, a helmet, a hat, etc.), as such the support structures 28 may include head-mounted support structures (e.g., a helmet housing, head straps, temples in a pair of eyeglasses, goggle housing structures, and/or other head-mounted structures). The support structures 28 may be worn on a head of a user during operation of the electronic device 10 and may support the input-output devices 24, the optical system 26, other components, one or more additional components of the electronic device 10.

### Optical Systems of Electronic Devices

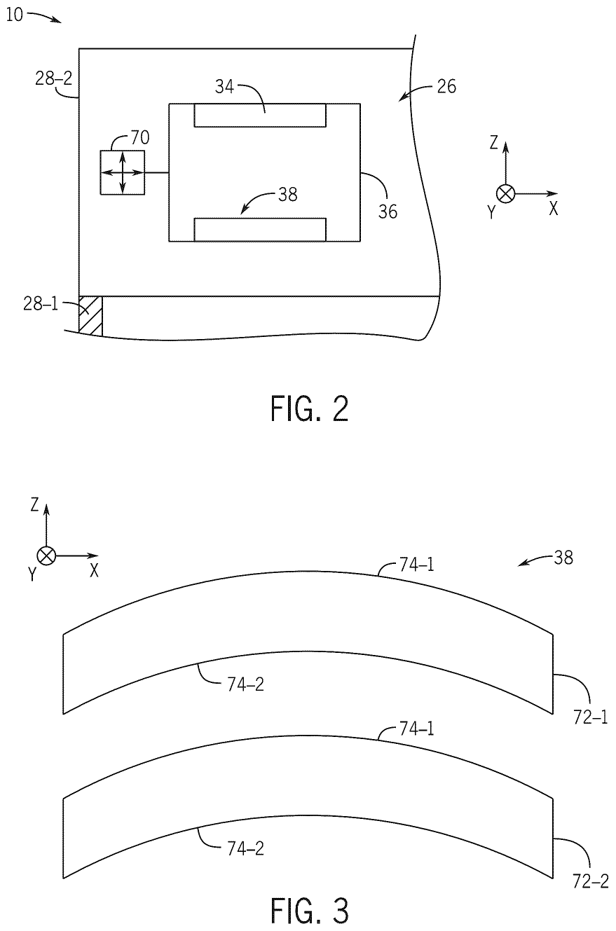

FIG. 2 is a schematic diagram of a top view of the electronic device 10 of FIG. 1 in an illustrative configuration in which the electronic device 10 is a head-mounted device. As shown in FIG. 2, the electronic device 10 may include the one or more support structures 28 of FIG. 1. The support structures may be as housing for the components of the electronic device 10 and may be used to mount the electronic device 10 onto a user's head. The support structures 28 may include, for example, straps or other supplemental support structures such as support structures 28-1 and structures that form housing walls such as support structure 28-2 (e.g., exterior housing walls, lens module structures, etc.).

As shown in FIG. 2, the electronic device 10 may include an optical system 26 as described in reference to FIG. 1. The electronic device 10 may include a left and a right optical modules that correspond respectively to a user's left eye and right eye. An optical module 36 corresponding to the user's left eye is shown in FIG. 2. The optical module 36 includes a corresponding lens module 38 as described in FIG. 1) and a positioner 70. The lens module 38 may include one or more lens elements arranged along a common axis. Each lens element may have any desired shape and may be formed from any desired material (e.g., glass, a polymer material such as polycarbonate or acrylic, a crystal such as sapphire, etc.) with any desired refractive index. The lens elements may have unique shapes and refractive indices that, in combination, focus light (e.g., from a display or from the physical environment) in a desired manner.

The optical module 36 may optionally be individually positioned relative to the user's eyes and relative to some of the housing wall structures of main unit 28-2 using positioning circuitry such as the positioner 70. Positioner 70 may include stepper motors, piezoelectric actuators, motors, linear electromagnetic actuators, shape memory alloys (SMAs), and/or other electronic components for adjusting the position of displays, the optical modules 36, and/or lens modules 38. The positioner 70 may be controlled by the control circuitry 18 of the electronic device 10 during operation. For example, the positioner 70 may be used to adjust the spacing between optical modules (and therefore the lens-to-lens spacing between the left and right lenses of right and left optical modules) to match the interpupillary distance (IPD) of a user's eyes. In another example, the lens module 38 may include an adjustable lens element. The curvature of the adjustable lens element may be adjusted in real time by the positioner 70 to compensate for a user's eyesight and/or viewing conditions. The electronic device 10 may optionally include a display positioned within the optical system 26 such as display 34 of FIG. 1.

FIG. 3 is a schematic diagram of a cross-sectional side view of a lens module 38 of FIG. 2 including one or more lens elements 72. As shown, the lens module 38 includes a first lens element 72-1 and a second lens element 72-2. The lens elements 72 may include converging lens (e.g., double convex, plano-convex, converging meniscus), diverging lens (e.g., double concave, plano-concave, plano-concave, diverging meniscus). One or more lens surfaces 74 of the lens elements 72 may have any desired curvature. For example, the lens surface 74 of the lens elements 72 may be a convex surface (e.g., a spherically convex surface, a cylindrically convex surface, or an aspherically convex surface), a concave surface (e.g., a spherically concave surface, a cylindrically concave surface, or an aspherically concave surface), or a freeform surface. A spherically curved surface (e.g., a spherically convex or spherically concave surface) may have a constant radius of curvature across the surface. In contrast, an aspherically curved surface (e.g., an aspheric concave surface or an aspheric convex surface) may have a varying radius of curvature across the surface. A cylindrical surface may only be curved about one axis instead of about multiple axes as with the spherical surface. In some cases, one of the lens surfaces 74 may have an aspheric surface that changes from being convex (e.g., at the center) to concave (e.g., at the edges) at different positions on the surface. This type of surface may be referred to as an aspheric surface, a primarily convex (e.g., the majority of the surface is convex and/or the surface is convex at its center) aspheric surface, a freeform surface, and/or a primarily convex (e.g., the majority of the surface is convex and/or the surface is convex at its center) freeform surface. A freeform surface may include both convex and concave portions. Alternatively, a freeform surface may have varying convex curvatures or varying concave curvatures (e.g., different portions with different radii of curvature, portions with curvature in one direction and different portions with curvature in two directions, etc.). Herein, a freeform surface that is primarily convex (e.g., the majority of the surface is convex and/or the surface is convex at its center) may sometimes still be referred to as a convex surface and a freeform surface that is primarily concave (e.g., the majority of the surface is concave and/or the surface is concave at its center) may sometimes still be referred to as a concave surface. In one example, shown in FIG. 3, the lens element 72-1 has a convex surface 74-1 that faces display 34 and an opposing concave surface 74-2. Lens element 72-2 has a convex surface 74-1 that faces lens element 72-1 and an opposing concave surface 74-2.

In some embodiments, one or both lens elements 72-1 and 72-2 may be adjustable. In one example, lens element 72-1 is a fixed (e.g., non-adjustable) lens element whereas lens element 72-2 is an adjustable lens element. The adjustable lens element 72-2 may be used to accommodate a user's eyeglass prescription, for example. The shape of lens element 72-2 may be adjusted if a user's eyeglass prescription changes (without replacing any of the other components within device 10). As another possible use case, a first user with a first eyeglass prescription (or no eyeglass prescription) may use device 10 with lens element 72-2 having a first shape and a second, different user with a second eyeglass prescription may use device 10 with lens element 72-2 having a second shape that is different than the first shape. Lens element 72-2 may have varying lens power and/or may provide varying amount of astigmatism correction to provide prescription correction for the user.

The example of lens module 38 including two lens elements is merely illustrative. In general, lens module 38 may include any desired number of lens elements (e.g., one, two, three, four, more than four, etc.). Any subset or all of the lens elements may optionally be adjustable. Any of the adjustable lens elements in the lens module 38 may optionally be fluid-filled lenses 40 (e.g., fluid-filled adjustable lens). The lens module 38 may also include any desired additional optical layers (e.g., partially reflective mirrors that reflect 50% of incident light, linear polarizers, retarders such as quarter wave plates, reflective polarizers, circular polarizers, reflective circular polarizers, etc.) to manipulate light that passes through lens module.

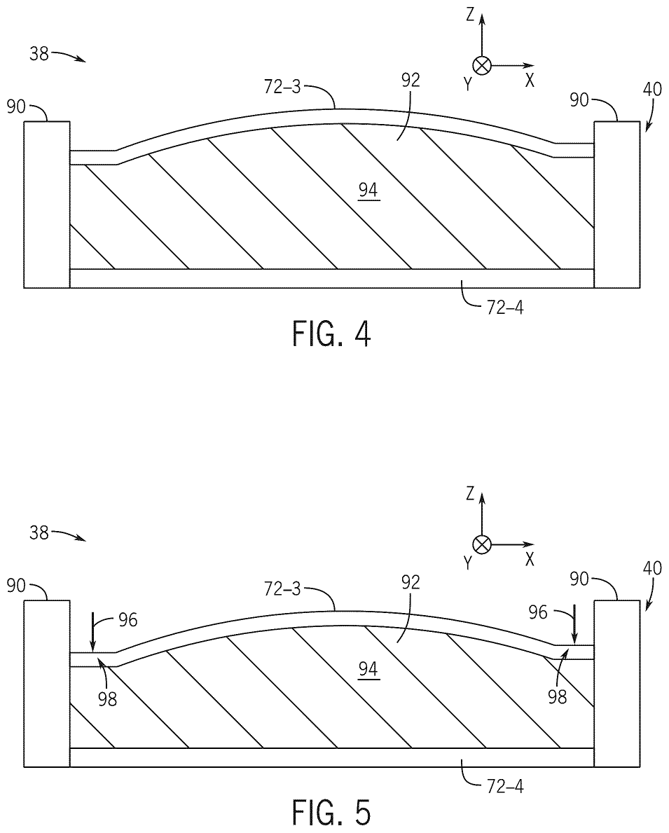

FIG. 4 is a cross-sectional side view of a lens module 38 including a fluid-filled lens 40. As shown, the lens module 38 may include a lens housing 90 used to define a fluid-filled chamber 94 of the fluid-filled lens 40. The lens module 38 may also include an optical fluid 92 and the fluid-filled chamber 94. The fluid-filled chamber 94 is interposed between a first lens elements 72-3 and a second lens element 72-4 and holds the optical fluid 92. The optical fluid 92 may be a liquid, gel, or gas with a pre-determined index of refraction (and may therefore sometimes be referred to as liquid 92, gel 92, or gas 92). The optical fluid 92 may be referred to as an index-matching oil, an optical oil, an optical fluid, an index-matching material, an index-matching liquid, etc. The lens elements 72 may have the same index of refraction or may have different indices of refraction. The optical fluid 92 that fills the fluid-filled chamber 94 may have an index of refraction that is the same as the index of refraction of the first lens element 72-3 but different from the index of refraction of the second lens element 72-4. In some instances, the optical fluid 92 may have an index of refraction that is the same as the index of refraction of the second lens element 72-4 but different from the index of refraction of the first lens element 72-3. In an embodiment, the optical fluid 92 may have an index of refraction that is the same as the index of refraction of the first lens element 72-3 and the second lens element 72-4, or may have an index of refraction that is different from the index of refraction of the first lens element 72-3 and the second lens element 72-4. Lens elements 72 may have a circular footprint, an elliptical footprint, or a footprint any another desired shape (e.g., an irregular footprint).

The amount of optical fluid 92 in the fluid-filled chamber 94 may have a constant volume or an adjustable volume. In embodiments, in which the amount of the optical fluid 92 is adjustable, the lens module 38 may also include a fluid reservoir and a fluid controlling component (e.g., a pump, stepper motor, piezoelectric actuator, motor, linear electromagnetic actuator, and/or other electronic component that applies a force to the fluid in the fluid reservoir) for selectively transferring fluid between the fluid reservoir and the fluid-filled chamber 94.

The lens elements 72 (e.g., the first lens element 72-3, the second lens element 72-4) may be transparent lens elements formed from any desired material (e.g., glass, a polymer material such as polycarbonate or acrylic, a crystal such as sapphire, etc.). The lens elements 72 may be elastomeric, semi-rigid, or rigid. For example, in some embodiments, the lens elements 72 may be elastomeric lens elements formed from a natural or synthetic polymer that has a low Young's modulus for high flexibility. The elastomeric lens element may be formed from a material having a Young's modulus of less than 1 GPa, less than 0.5 GPa, less than 0.1 GPa, etc. The elastomeric lens element may be flexible along a first axis even when the lens element 72 is curved along a second axis perpendicular to the first axis. In certain embodiments, the lens elements 72 may be semi-rigid lens elements formed from a semi-rigid material (e.g., polycarbonate, polyethylene terephthalate (PET), polymethylmethacrylate (PMMA), acrylic, glass, or any other desired material) that is stiff and solid, but not inflexible. Semi-rigid lens element may, for example, be formed from a thin layer of polymer or glass having a Young's modulus that is greater than 1 GPa, greater than 2 GPa, greater than 3 GPa, greater than 10 GPa, greater than 25 GPa, etc. The properties of semi-rigid lens elements may result in the lens element becoming rigid along a first axis when the lens element 72 is curved along a second axis perpendicular to the first axis or, more generally, for the product of the curvature along its two principal axes of curvature to remain roughly constant as it flexes. The properties of semi-rigid lens elements may allow the semi-rigid lens elements to form a cylindrical lens with tunable lens power and a tunable axis.

In some embodiments, the lens elements 72 may be rigid lens elements formed from glass, a polymer material such as polycarbonate or acrylic, a crystal such as sapphire, etc. In general, the rigid lens elements may not deform when pressure is applied to the lens elements 72 within the lens module 38. In other words, the shape and position of the rigid lens elements may be fixed. Each surface of a rigid lens element may be planar, concave (e.g., spherically, aspherically, or cylindrically concave), or convex (e.g., spherically, aspherically, or cylindrically convex). Rigid lens elements may be formed from a material having a Young's modulus that is greater than greater than 25 GPa, greater than 30 GPa, greater than 40 GPa, greater than 50 GPa, etc.

FIG. 5 is a schematic embodiment of a cross-sectional side view of the lens module 38 and the fluid-filled lens 40 of FIG. 4 including an illustrative adjustment of a shape of the first lens element 72-3. As shown, during adjustments of the lens module 38, the first lens element 72-3 may be biased in direction 96 at one or more points 98 along its periphery (e.g., a point force is applied in direction 96 at multiple points). In this way, a curvature of the first lens element 72-3 (and accordingly, the lens power of the first lens element 72-3) may be adjusted.

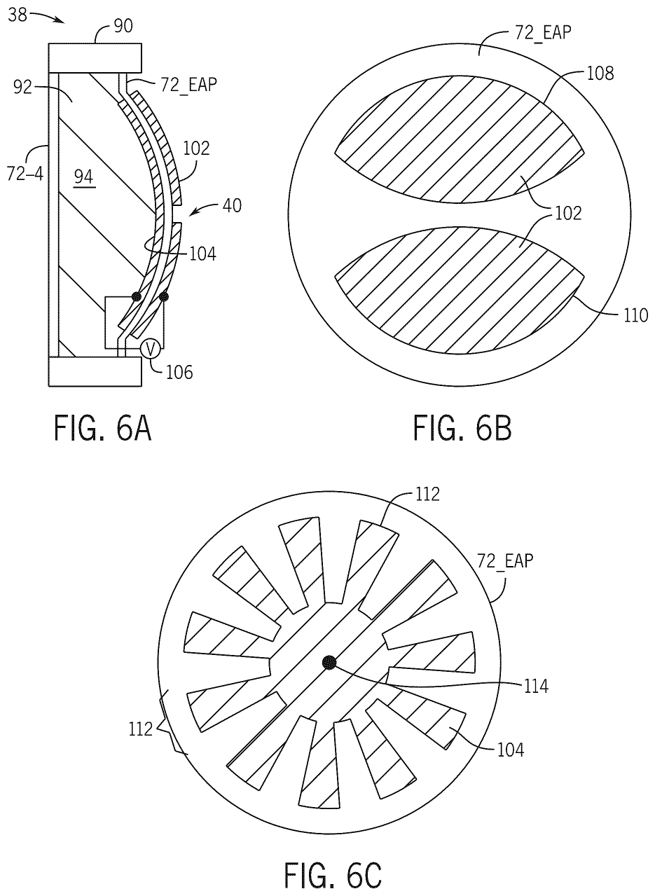

In some embodiments, gravity induced aberrations/sagging may be mitigated using an optical module with actuatable stiffness. FIGS. 6A-6C illustrate a lens module having a membrane with adjustable stiffness. FIG. 6A is a schematic diagram of a cross-sectional side view of the lens module 38 and the fluid-filled lens 40 of FIG. 4 including a lens element 72-EAP formed by an electroactive polymer (EAP), according to embodiments of the present disclosure. The EAP lens element 72-EAP is a material with a property that changes in response to an electric field. The EAP lens element 72-EAP may have an increased stiffness in response to an electric field across the material (relative to when no electric field is applied across the material). One or more patterned electrodes may be formed on either side of EAP lens element 72-EAP to dynamically adjust the electric field applied across EAP lens element 72-EAP, which accordingly dynamically adjusts the stiffness of EAP lens element 72-EAP. The EAP lens element 72-EAP is interposed between a first electrode 102 on a first side of the lens element and a second electrode 104 on a second side of the EAP lens element 72-EAP. The first and second electrode 102, 104 may be formed from a transparent conductive material such as indium tin oxide (ITO) or any other desired material.

In some embodiments, voltages may be applied to the first and second electrode 102, 104. A voltage difference 106 may be created between the first electrode 102 and the second electrode 104. The voltage difference 106 may modify one or more properties of the EAP lens element 72-EAP. For example, control circuitry of the lens module 38 may control the voltage difference 106 applied between the first and second electrode 102, 104 to control the stiffness of the EAP lens element 72-EAP. In some embodiments, the control circuitry may sense an orientation of the EAP lens element 72-EAP and control the voltage difference to adjust the stiffness of the EAP lens element 72-EAP to compensate for sagging caused by gravity at the orientation.

FIG. 6B is a schematic diagram of the EAP lens element 72-EAP of FIG. 6A, according to embodiments of the present disclosure. The EAP lens element 72-EAP may include the electrode 102. The electrode 102 may be formed from an electrode pattern made up of a first electrode pattern 108 and a second electrode pattern 110. The first and second electrode pattern 108, 110 may be formed from similar or different electrode materials. FIG. 6C is a schematic diagram of the EAP lens element 72-EAP of FIG. 6A including one or more fingers 112 patterned into the second electrode 104, according to embodiments of the present disclosure. The fingers 112 may extend radially outward from a center 114 of the EAP lens element 72-EAP. Different voltages may be applied to the electrode 102 and 104 of FIG. 6A. The pattern of electrode 104 in FIG. 6C may create a varying voltage profile with different voltages at different fingers 112 The resulting stiffness variations in the EAP lens element 72-EAP may mitigate undesired gravity sagging in the lens module 38.

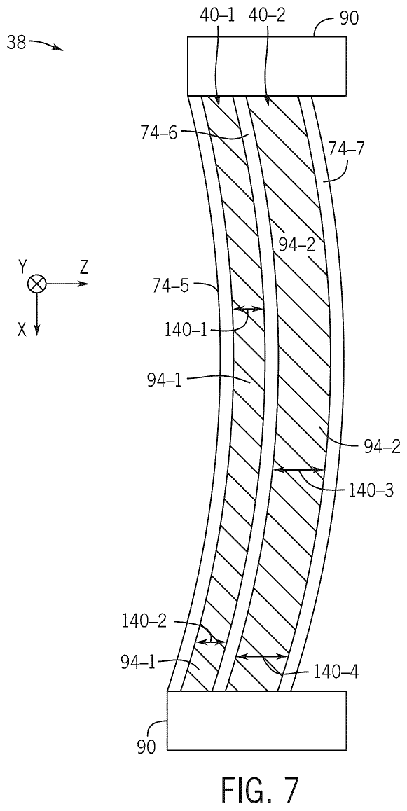

FIG. 7 is a schematic embodiment of a cross-sectional side view of a lens module 38 including a first fluid-filled chamber 94-1 and a second fluid-filled chamber 94-2. The lens module 38 of FIG. 7 includes a first fluid-filled lens 40-1 and a second fluid-filled lens 40-2. The first fluid-filled lens 40-1 includes the first fluid-filled chamber 94-1 formed between a first lens element 74-5 and a second lens element 74-6. The second fluid-filled lens 40-2 includes the second fluid-filled chamber 94-2 formed between the second lens element 74-6 and a third lens element 74-7. The lens module 38 includes a lens housing 90 used to define the first and second fluid-filled chamber 94-1, 94-2 of the first and second fluid-filled lenses 40-1, 40-2. The first fluid-filled chamber 94-1 includes a first optical fluid 92-1. The second fluid-filled chamber 94-2 includes a second optical fluid 92-2. In some embodiments, the lens housing 90 may include one or more actuators. The actuators may manipulate the second lens element 74-6 and/or the third lens element 74-7 to change a shape configuration of the first and/or second fluid-filled lenses 40-1, 40-2. In certain embodiments, neither the second lens element 74-6 and/or the third lens element 74-7 may be manipulated during operation of the lens module 38.

In some embodiments, a thickness across an aperture height of each fluid-filled chamber 94 may be affected by gravity causing a portion of the lens module 38 to sag. As shown in FIG. 7, the first fluid-filled chamber 94-1 has a first thickness 140-1 at a first position within the first fluid-filled chamber 94-1 and a second thickness 140-2 at a second position within the first fluid-filled chamber 94-1. Due to a bulge caused by gravity, the thickness 96-2 may be greater than the thickness 96-1. The second fluid-filled chamber 94-2 has a first thickness 140-3 at a first position within the chamber and a second thickness 140-4 at a second position within the second fluid-filled chamber 94-2. Due to a bulge caused by gravity, thickness 140-4 may be greater than thickness 140-3. It should be noted, a position of the bulge in each of the fluid-filled chambers 94 may vary depending on relative densities of the optical fluids 92 within the fluid-filled chambers 94.

The properties of the first and second optical fluid 92-1, 92-2, and the first, second, and third lens elements 74-5, 74-6, and 74-7 may be selected such that sag due to gravity in the second fluid-filled chamber 94-2 is compensated by sag due to gravity in the first fluid-filled chamber 94-1. In other words, the first and second fluid-filled chambers 94-1, 94-2 compensate one another to mitigate optical aberrations otherwise caused by gravity effects.

In some embodiments, the first lens element 74-5 may be a rigid lens element and the second and third lens elements 74-6, 76-7 may be elastomeric lens elements. The first lens element 74-5 has a convex surface facing the second lens element 74-6 and a concave surface facing a user (when the device is worn by the user). The third lens element 74-7 has a convex surface facing the second lens element 74-6 and a concave surface facing the first lend element 74-5. The third lens element 74-7 has a convex surface facing away from the user and a concave surface facing the second lens element 74-6. It should be noted, the illustrated example is merely illustrative and in general each lens elements may be rigid, elastomeric, or semi-rigid and may have any desired shape.

In certain embodiments, the first and second optical fluids 92-1, 92-2 may be materials with properties that are selected such that the first and second fluid-filled chambers 94-1, 94-2 mutually compensate one another for gravity induced aberrations. The first and second optical fluids 92-1, 92-2 may have different refractive indices and densities. As one example, the first optical fluid 92-1 may have a refractive index of 1.35 whereas the second optical fluid 92-2 may have a refractive index of 1.50. In this example, the difference in refractive index between the first and second optical fluids 92-1, 92-2 is 0.15 This example is merely illustrative. In general, the difference in refractive index between first and second optical fluids 92-1, 92-2 may be at least 0.05, at least 0.10, at least 0.15, at least 0.20, less than 0.50, less than 0.40, less than 0.30, etc.

As another example, the first optical fluid 92-1 may have a density of 1.45 g/cm³ whereas the second optical fluid 92-2 may have a density of 1.15 g/cm³. In this example, the difference in density between the first and second optical fluids 92-1, 92-2 is 0.3 g/cm³. This example is merely illustrative. In general, the difference in density between the first and second optical fluids 92-1, 92-2 may be at least 0.30 g/cm³, at least 0.50 g/cm³, at least 0.70 g/cm³, at least 0.90 g/cm³, at least 1.10 g/cm³, less than 1.50 g/cm³, less than 1.30 g/cm³, less than 1.10 g/cm³, less than 1.00 g/cm³, etc. In the aforementioned examples, the first optical fluid 92-1 has a higher density and lower refractive index than the second optical fluid 92-2. This example is merely illustrative and the second optical fluid 92-2 may instead optionally have a higher density and/or lower refractive index than the first optical fluid 92-1.

### Optical Fluids and Systems

As described above, the optical fluids may include PFAS-free fluids for use in lens modules of electronic devices. A first optical fluid may have a high density and low refractive index whereas a second optical fluid may have a low density and high refractive index. Examples of the optical fluids are described in detail below. In some embodiments, the optical fluid includes one or more low index fluids (e.g., low refractive index fluid). The low index fluids may include a modified polyether material, a modified polysiloxane material, a modified polyester material, and the like. In some embodiments, the optical fluid includes one or more low high fluids (e.g., high refractive index fluid). The high index fluids may include a modified polysulfide material, a modified polypropylene material, and the like.

The brackets in the below formulas represent repeating units. For example, there may be n repeating units of the structure inside the corresponding parenthesis. The denotation n ranges from 1 to 100. It is noted that n is an average and the polyether material may have a distribution having an average value of n. The modified polyether material may have a relatively low refractive index and a relatively high density.

In some embodiments, the low index fluids may include the modified polyether material represented by formula (I).

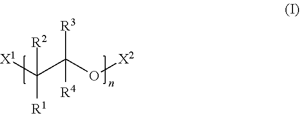

In certain embodiments, R¹, R², R³, and R⁴ are independently hydrogen, fluorine, a fluoromethyl group, a difluoromethyl group, a pentafluorosulfanyl group, a pentafluorosulfanyl methyl group, or CH₂—O—Y. Y is chosen from R⁵, R⁶, or 1,3-bis(trifluoromethoxy) propane, or a trifluoromethyl group. R⁵ is an aromatic group represented by formula (II),

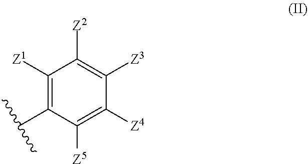

wherein Z¹⁻⁵ are independently hydrogen, fluorine, R⁶, or O—R⁶. R⁶ is a C₁-C₈ substituted hydrocarbyl wherein the hydrocarbyl is substituted with one or more fluorine, a fluoromethyl group, a difluoromethyl group, a trifluoromethoxy group, or a pentafluorosulfanyl group. For example, R⁶ may include (CH₂)_{w}CF₂H, (CH₂)_{w}O(CH₂)_{w}CF₂H, (CH₂)_{w}CFH₂, (CH₂)_{w}O(CH₂)_{w}′CFH₂, (CH₂)_{w}O(CH₂)_{w}′SF₅, (CH₂)_{w}SF₅, (CH₂)_{w}OCF₃, or (CH₂)_{w}O(CH₂)_{w}′OCF₃, (CH₂)_{w}CH₃, or (CH₂)_{w}O(CH₂)_{w}CH₃ where w ranges from 0 to 8 and w′ ranges from 0 to 8.

In some embodiments, R¹ and R² of formula I are independently hydrogen or a trifluoromethoxy methyl group and R³ and R⁴ are independently hydrogen, a methyl group, a trifluoromethoxy methyl group, a difluoromethyl group, a 1-ethoxy-2,3,4,5,6-pentafluorophenyl, (2-ethoxyethyl) pentafluoro sulfane group, a pentafluorosulfanyl group, and/or pentafluorosulfanyl methyl group.

X¹ and X² are independently hydrogen, fluorine, chlorine, bromine, a hydroxyl group, a methyl group, a methylpropane, R⁵, O—R⁵, S—R⁵, R⁶, O—R⁶, or S—R⁶. Further in some embodiments, X¹ and X² are independently hydrogen, fluorine, chlorine, bromine, a hydroxyl group, a methyl group, a methylpropane, a 2-trifluoromethoxy ethoxy group, a 2,2-difluoroethoxy group, 2-(pentafluorosulfanyl)ethoxy group, or a pentafluorophenoxy group.

In some embodiments, modified polyether materials and/or one or more additional types of optical fluids may be synthesized using one or more precursor compounds. The precursor compounds may include an allyl trifluoromethyl ether, represented by precursor P1 (formula P1), a 2-[(trifluoromethoxy)methyl]oxirane, represented by precursor P2 (formula P2), a 2-(pentafluorophenoxymethyl)oxirane, represented by precursor P3 (formula P3), a 1,4-bis(trifluoromethoxy)-2-butene, represented by precursor P4 (formula P4), a 2,3-bis((trifluoromethoxy)methyl)oxirane, represented by precursor P5 (formula P5), a 2-((2-(pentafluoro-2⁶-sulfaneyl)ethoxy)methyl)oxirane, represented by precursor P6 (formula P6), and/or one or more additional compounds.

Precursor P1, the allyl trifluoromethyl ether, represented by formula P1, is formed by a reaction where 2,4-dinitro-1-trifluoromethoxybenzene (e.g., reagent R1) is reacted with 4-(dimethylamino)pyridine (reagent R2) to form the precursor P1. The example reaction scheme is shown below:

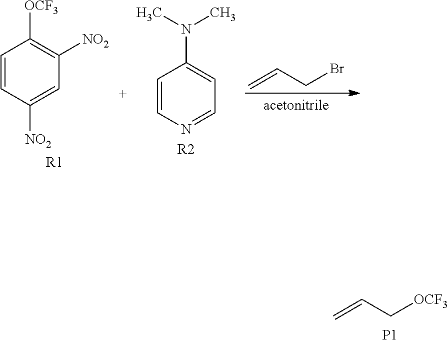

The reaction to form precursor P1 includes charging a 5 L 4-neck flask with a reflux condenser and overhead mechanical stirrer under an inert atmosphere of nitrogen with anhydrous acetonitrile (1.0 L) and 2,4-dinitro-1-trifluoromethoxybenzene (reagent R1, 597 g). 4-(dimethylamino)pyridine (reagent R2, 258 g) is then added portion wise while cooling the reaction flask in an ice bath to form a mixture. The mixture stirs for about 30 min and then allyl bromide (307 g) is added in one portion. The reaction is heated at 45° C. for about 62 h. The reaction flask is fitted with a distillation apparatus and then the reaction flask was heated to effect distillation of the product as well as unreacted allyl bromide and acetonitrile solvent. A crude product (353 g) was then subjected to a fine distillation using a concentric tube distillation column. Vapors collected with a boiling point between 15° C. and 24° C. were collected as high purity allyl trifluoromethyl ether (precursor P1, 224 g, 84%). Chemical Formula: C₄H₅F₃O. Characterization of precursor P1: ¹H NMR (300 MHz, CDCl₃) δ 5.82-6.02 (m, 1H), 5.40 (dq, J=17.1, 1.4 Hz, 1H), 5.32 (dq, J=10.5, 1.2 Hz, 1H), 4.46 (ddd, 5.8, 1.5, 1.3 Hz, 2H). ¹⁹F NMR (282 MHz, CDCl₃): δ -60.95 (s, 3F). GCMS: Expected for C₄H₅F₃O: m/z=126.0. Measured: m/z=126.1.

Precursor P2, the 2-[(trifluoromethoxy)methyl]oxirane, represented by formula P2, is formed by a reaction of precursor P1 (e.g., allyl trifluoromethyl ether) with methylene chloride and 3-chloroperbenzoic acid (mCPBA). The example reaction scheme is shown below:

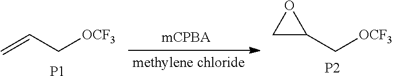

The reaction to form precursor P2 includes charging a dry 1 L 3-neck flask with a reflux condenser and magnetic stir bar under an inert atmosphere of nitrogen with methylene chloride (530 g), allyl trifluoromethyl ether (precursor p1, 80 g) and 3-chloroperbenzoic acid (175 g). The flask was heated at between about 31° C. and 35° C. for 64 h. The reaction contents were transferred into a stirring solution of saturated aqueous sodium bicarbonate (400 mL) and allowed to stir for about 1 h. The mixture was phase separated, and the product mixture was then washed with saturated aqueous sodium bicarbonate (400 mL), and saturated aqueous sodium chloride (400 mL). The product mixture was then dried by stirring with anhydrous magnesium sulfate (20 g) for about 2 h, and then filtered into a 3-neck flask. The crude product mixture was subjected to distillation using a concentric tube distillation column under an atmosphere of nitrogen. The vapors collected with a boiling point of about 90° C. to 91° C. were collected as high purity 2-[(trifluoromethoxy)methyl]oxirane (precursor 2, 44.7 g, 50%). Chemical formula: C₄H₅F₃O₂. Characterization of precursor P2: ¹H NMR (300 MHz, CDCl₃) δ 4.16 (dd, J=11.5, 3.3 Hz, 1H), 3.88 (dd, J=11.5, 6.1 Hz, 1H), 3.19-3.28 (m, 1H), 2.87 (dd, J=4.7, 4.2 Hz, 1H), 2.66 (dd, J=4.8, 2.6 Hz, 1H). ¹⁹F NMR (282 MHZ, CDCl₃): δ-61.47 (s, 3F). GCMS: Expected for C₄H₅F₃O₂: m/z=142.0. Measured: m/z=112.0 [M−CH₂O].

Precursor P3, the 2-(pentafluorophenoxymethyl)oxirane, represented by formula P3, is formed by a reaction of anhydrous potassium carbonate with pentafluorophenol (reagent R3) and 1-chloro-2,3, -epoxypropane (reagent R4). The example reaction scheme is shown below:

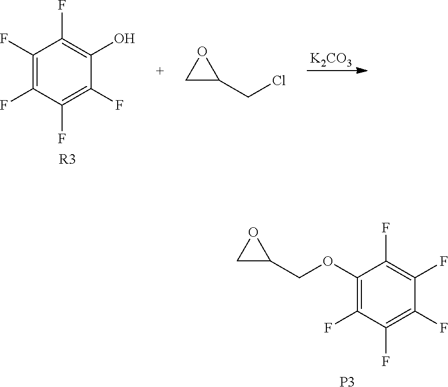

The reaction to form precursor P3 includes charging anhydrous potassium carbonate (27.6 g) was charged into a 250 mL 3-neck flask fitted with a reflux condenser under an inert atmosphere of nitrogen. Then pentafluorophenol (Reagent R³, 25 g) 1-chloro-2,3-epoxypropane (93 g) and were added. The mixture was heated at 100° C. for about 14 h, and then cooled. The mixture was filtered into a round bottom flask and then subjected to distillation. The vapors collected with a boiling point of about 68° C. to 70° C./1.5 torr were collected as high purity 2-(pentafluorophenoxymethyl)oxirane (precursor P3, 25 g, 77%). Chemical formula: C₉H₅F₅O₂ Characterization of precursor P3: ¹H NMR (60 MHz, CDCl₃) δ 4.46 (dd, J=11.5, 2.9 Hz, 1H), 4.00 (dd, J=11.5, 6.1 Hz, 1H), 3.50-3.15 (m, 1H), 3.00-2.55 (m, 2H). ¹⁹F NMR (56 MHZ, CDCl₃): δ-156.3-−157.6 (m, 2F), −162.7-−164.5 (m, 3F). GCMS: Expected for C₉H₅F₅O₂: m/z=240.0. Measured: m/z=240.1.

Precursor P4, the 1,4-bis(trifluoromethoxy)-2-butene, represented by formula P4, is formed by a reaction of 2,4-dinitro-1-trifluoromethoxybenzene (e.g., reagent R1) is reacted with 4-(dimethylamino)pyridine (reagent R2). The example reaction scheme is shown below:

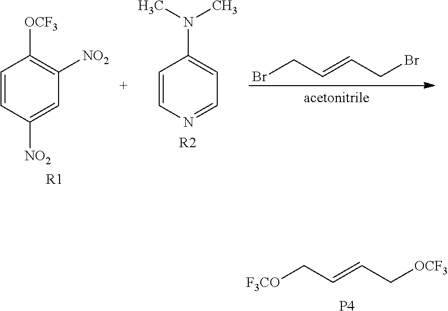

The reaction to form precursor P4 includes charging a 0.5 L 4-neck flask with a reflux condenser and overhead mechanical stirrer under an inert atmosphere of nitrogen with anhydrous acetonitrile (0.5 L) and 2,4-dinitro-1-trifluoromethoxybenzene (reagent R1, 165 g). 4-(dimethylamino)pyridine (reagent R2, 72 g) is then added portion wise while cooling the reaction flask in an ice bath to form a mixture. The mixture stirs for about 30 min and then trans-1,4-dibromo-2-butene (50 g) was converted into 1,4-bis(trifluoromethoxy)-2-butene by heating the mixture at 45° C. for about 92 h. The target compound was subjected to distillation using a concentric tube distillation column. The vapors collected with a boiling point of about 48° C. to 50° C. at a pressure of 50 torr were collected as high purity 1,4-bis(trifluoromethoxy)-2-butene (precursor P4, 74%). Chemical Formula: C₆H₆F₆O₂. Characterization of precursor P4: ¹H NMR (300 MHz, CDCl₃) δ 6.00-5.80 (m, 2H), 4.58-4.39 (m, 4H). ¹⁹F NMR (282 MHZ, CDCl₃): δ-61.16 (s, 6F). ¹³C NMR (75.5 MHz, CDCl₃) δ 127.4 (s), 121.8 (q, J=256 Hz), 66.4 (q, J=3.6 Hz). GCMS: Expected for C₆H₆F₆O₂: m/z=224.0. Measured: m/z=224.0.

Precursor P5, the 2,3-bis((trifluoromethoxy)methyl)oxirane, represented by formula P5, is formed by a reaction of precursor P4 (e.g., 1,4-bis(trifluoromethoxy)-2-butene) with methylene chloride and 3-chloroperbenzoic acid (mCPBA). The example reaction scheme is shown below:

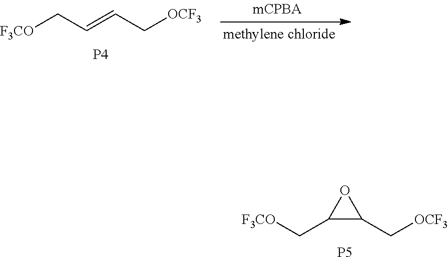

The reaction to form precursor P5 includes adding 3-chloroperbenzoic acid (45.2 g), methylene chloride (120 mL), and 1,4-bis(trifluoromethoxy)-2-butene (precursor P4, 36.7 g) to a 420 mL glass pressure vessel. The vessel was sealed and then heated at 65° C. for about 40 h. The reaction was cooled and then the contents were added to saturated aqueous sodium bicarbonate (300 mL) and the mixture was allowed to stir for about 60 min. The layers are phase separated and then the aqueous phase was washed with methylene chloride (100 mL) twice. The combined organics were washed with aqueous sodium bicarbonate (300 mL) twice, and then saturated aqueous sodium chloride (300 mL). The organic phase was dried by stirring with anhydrous magnesium sulfate for about 4 h. The mixture was filtered into a round bottom flask and then subjected to distillation using a concentric tube column. The vapors collected with a boiling point of about 53° C. to 54° C./10 torr were collected as high purity 2,3-bis((trifluoromethoxy)methyl)oxirane (precursor P5, 25.4 g, 68%). Characterization of precursor P5: ¹H NMR (300 MHZ, CDCl₃) δ 4.17 (dd, J=11.7, 3.1 Hz, 2H), 3.98 (dd, J=11.7, 5.2 Hz, 2H), 3.22 (m, 2H). ¹⁹F NMR (282 MHz, CDCl₃): −61.65 (s, 6F).

Precursor P6, the 2-((2-(pentafluoro-λ⁶-sulfaneyl)ethoxy)methyl)oxirane, represented by formula P6, is formed by a reaction of 2-(pentafluorosulfanyl)ethan-1-ol (reagent R5) with 1-chloro-2,3-epoxypropane (reagent R4) to form 1-chloro-3-(2-(pentafluoro-λ⁶-sulfaneyl)ethoxy) propan-2-ol (reagent R6). form 1-chloro-3-(2-(pentafluoro-λ⁶-sulfaneyl)ethoxy) propan-2-ol (reagent R6) was further reacted with sodium hydroxide to form precursor P6. The example reaction scheme is shown below:

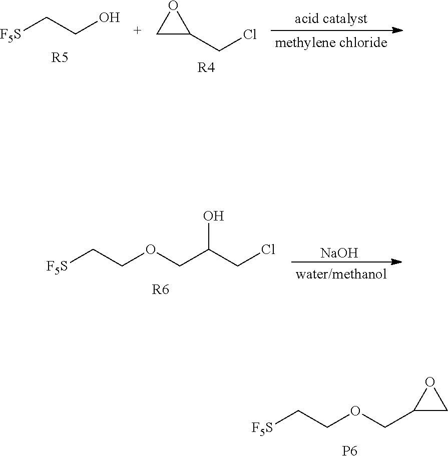

The reaction to form 1-chloro-3-(2-(pentafluoro-λ⁶-sulfaneyl)ethoxy) propan-2-ol (reagent R6) includes combining anhydrous methylene chloride (20 mL) and an acid catalyst (0.60 g) in an 100 mL flask under an inert atmosphere of nitrogen. The mixture was cooled in an ice bath and then 2-(pentafluorosulfanyl)ethan-1-ol (reagent R5, 20.0 g) was added, and then 1-chloro-2,3-epoxypropane (reagent R4, 10.8 g) was added dropwise over 1 h. The mixture was stirred at 0° C. for about 3 h and then about 16 h at 20° C. The reaction flask was fitted with a distillation column and then 1-chloro-3-(2-(pentafluoro-λ⁶-sulfaneyl)ethoxy) propan-2-ol (reagent R6, 8.2 g) was collected with a vapor temperature of 46° C. to 54° C. at 0.5 torr. Chemical formula: C₅H₁₀ClF₅O₂S. Chemical characterization of R⁶: GCMS: Expected for C₅H₁₀ClF₅O₂S: m/z=264.0. Measured: m/z=228.0 [M-HCl].

The 1-chloro-3-(2-(pentafluoro-λ⁶-sulfaneyl)ethoxy) propan-2-ol (reagent R6, 8.2 g) was dissolved in methylene chloride (20 mL) and then the mixture was added dropwise to sodium hydroxide (1.24 g) dissolved in water (20 mL) and methanol (20 mL) cooled in an ice bath. The mixture stirred in the ice bath for about 5 min and then stirred at 20° C. for about 6 h. The mixture was poured into a separatory funnel and diluted with water (200 mL) and methylene chloride (100 mL). The layers were separated and then the organic layer was washed with water (200 mL), saturated aqueous sodium bicarbonate (200 mL), and saturated aqueous sodium chloride (200 mL). The organic phase was dried by stirring with anhydrous magnesium sulfate and then filtered to remove the solids. Solvents were evaporated on a rotary evaporator and then the target compound was isolated by distillation using a glass Vigreux column. The vapors collected with a boiling point of about 26° C. to 32° C. at 0.5 torr were collected as high purity 2-((2-(pentafluoro-λ⁶-sulfaneyl)ethoxy)methyl)oxirane (precursor P6, 6.41 g, 24%). Chemical formula: C₅H₉F₅O₂S. Chemical characterization of P6: ¹H NMR (300 MHz, CDCl₃) δ 4.00-3.72 (m, 5H), 3.37 (dd, J=11.7, 6.0 Hz, 1H), 3.17-3.08 (m, 1H), 2.79 (dd, J=5.0, 4.2 Hz, 1H), 2.60 (dd, J=5.0, 2.7 Hz, 1H). ¹⁹F NMR (282 MHz, CDCl₃): δ4.7-82.5 (m, 1F), 65.4 (dt, J=145, 7.9 Hz, 4F). GCMS: Expected for C₅H₉F₅O₂S: m/z=228.0. Measured: m/z=198.0 [M−CH₂O].

Compound L1 is an example the low index fluid including a modified polyether material, represented by formula (L1). Compound L1 is formed by reacting 2-(trifluoromethoxy)ethanol (reagent R7) and 2-[(trifluoromethoxy)methyl]oxirane (precursor P2). The example reaction scheme is shown below:

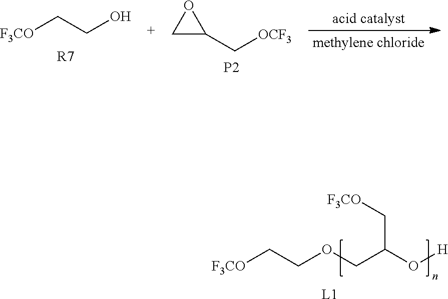

The reaction to form compound L1 includes combining anhydrous methylene chloride (80 mL) and an acid catalyst (1.4 g) in a 250 mL flask under an inert atmosphere of nitrogen. The mixture was cooled in an ice bath and then 2-(trifluoromethoxy)ethanol (reagent R7, 5.63 g) was added. Then, 2-[(trifluoromethoxy)methyl]oxirane (precursor P2, 80.0 g) was added dropwise over about 4 h. The mixture was stirred for an additional 60 min and then filtered through silica gel eluting with methylene chloride and ethyl acetate (95:5 v:v). Solvents were evaporated from the product mixture on a rotary evaporator and then a high vacuum line at 0.5 torr for about 2 h, while heating the product at 85° C. The residue was filtered through a 0.1 μm PTFE syringe filter to yield compound L1 as a clear and colorless viscous liquid (compound C₁, 79.4 g, 93%). Chemical characterization: 1H NMR (300 MHz, CDCl₃) δ 3.20-4.44 (m, 72.9H), 2.71 (s, 1H). ¹⁹F NMR (282 MHZ, CDCl₃): δ-61-−63 (m). Density measured at 20° C. is 1.454 g/mL. Optical characterization: refractive index measured using a Schmidt Haensch ATR L Dispersion Refractometer at 20° C.: 1.3694 at 593.0 nm.

Compound L2 is an example the low index fluid including a modified polyether material, represented by formula (L2). Compound L2 is formed by reacting potassium hydroxide, ethanol (reagent 8), and 2-(difluoromethyl)oxirane (reagent 9). The example reaction scheme is shown below:

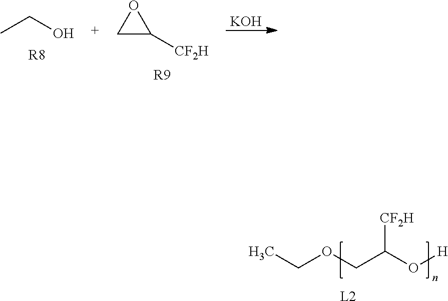

The reaction to form compound L2 includes combining potassium hydroxide (0.37 g), ethanol (0.98 g), and 2-(difluoromethyl)oxirane (20.0 g) in a 50 mL 3-neck flask under an inert atmosphere of nitrogen. The mixture was heated at 60° C. for about 14 h, then 75° C. for about 2.5 h, then 80° C. for about 1.5 h. NMR analysis indicates complete monomer conversion. The reaction contents were dissolved in methylene chloride (100 mL) and washed sequentially with 7% hydrochloric acid (65 mL), then saturated aqueous sodium chloride (60 mL). The organic mixture was then dried by stirring with anhydrous magnesium sulfate (5 g) for 60 min, then filtered to remove the salts. The methylene chloride solvent was evaporated on a rotary evaporator and then on a high vacuum line at 0.5 torr for about 2 h, while the product was heated at 85° C. The residue was filtered through a 0.1 μm PTFE syringe filter to L2 as a clear and colorless viscous fluid (19.0 g, 91%). Chemical characterization of L2: ¹H NMR (300 MHz, CDCl₃) δ 5.82 (t, J=55 Hz, 16.1H), 2.8-4.4 (m, 52.0H), 1.19 (t, J=7.0 Hz, 3H). ¹⁹F NMR (282 MHZ, CDCl₃): δ-133-−127 (m). Density measured using at 20° C. is 1.449 g/mL. Optical characterization: refractive index measured using a Schmidt Haensch ATR L Dispersion Refractometer at 20° C.: 1.4034 at 593.0 nm.

Compound L3 is an example the low index fluid including a modified polyether material, represented by formula (L3). Compound L3 is formed by reacting 2,2-difluoroethanol (reagent R10) with 2-(pentafluorophenoxymethyl)oxirane (precursor P3). The example reaction scheme is shown below:

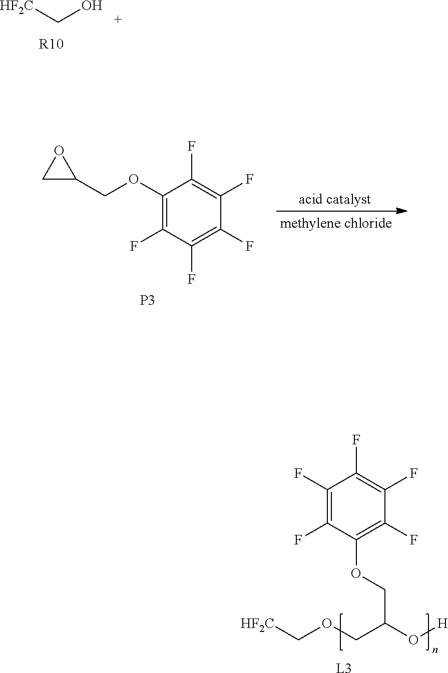

The reaction to form compound L3 includes combining anhydrous methylene chloride (10 mL) and an acid catalyst (0.04 g) in a 50 mL flask under an inert atmosphere of nitrogen. The mixture was cooled in an ice bath and then 2,2-difluoroethanol (reagent R10, 0.56 g) was added, and then 2-(pentafluorophenoxymethyl)oxirane (precursor P3, 10.0 g) was added dropwise over about 4 h. The mixture was stirred for an additional 14 h and then filtered through silica gel eluting with methylene chloride and ethyl acetate. Solvents were evaporated from the product mixture on a rotary evaporator and then a high vacuum line at 0.5 torr for about 2 h, while heating the product was heated at 85° C. The residue was filtered through a 0.45 μm syringe filter to yield compound L3 as a clear and colorless viscous liquid. Chemical composition of L3: ¹H NMR (300 MHZ, CDCl₃) δ 5.84 (t, J=56 Hz, 1H), 3.4-4.5 (m, 39.0H), 2.87 (m, 1H). ¹⁹F NMR (282 MHz, CDCl₃): δ-125-−127 (m, 2F), −157-−158 (m, 15.8F), −163-−164.5 (m, 23.4F). Density measured at 18° C. is 1.59 g/mL. Optical characterization: refractive index measured using a Schmidt Haensch ATR L Dispersion Refractometer at 20° C.: 1.47 at 589.0 nm.

Compound L4 is an example the low index fluid including a modified polyether material, represented by formula (L4). Compound L4 is formed by reacting 2,2-difluoroethanol (reagent R10) with 2,3-bis((trifluoromethoxy)methyl)oxirane (precursor P5). The example reaction scheme is shown below:

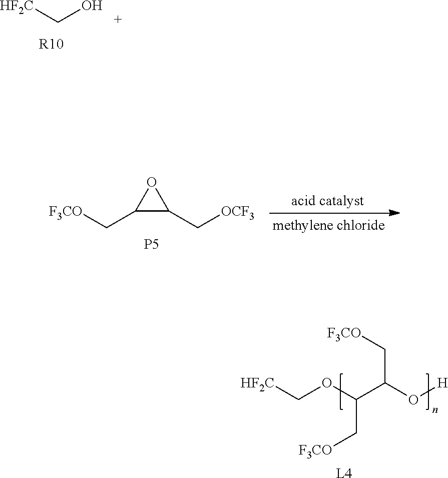

The reaction to form compound LA includes combining anhydrous methylene chloride (4 mL), 2,2-difluoroethanol (reagent R10, 0.276 g), and 2,3-bis((trifluoromethoxy)methyl)oxirane (precursor P5, 4.02 g) were combined in a 250 mL PFA 3-neck flask under an inert atmosphere of nitrogen. The flask was cooled to −30° C. and then an acid catalyst (27 μL) was added. The reaction was allowed to warm slowly to room temperature and stir for about 16 h. The reaction mixture was poured into saturated aqueous sodium bicarbonate (75 mL) and diluted with methylene chloride (75 mL). The layers were separated and then the organic phase was washed with saturated aqueous sodium chloride (75 mL). The organic phase was dried by stirring with anhydrous magnesium sulfate and then filtered to remove the solids. Solvents were removed on a rotary evaporator and then a high vacuum line at 0.5 torr for about 2 h, while heating the product was heated at 60° C. The residue was filtered through a 0.45 μm syringe filter to yield compound L4 (3.83 g) as a viscous liquid. Chemical composition of L4: ¹H NMR (300 MHz, CDCl₃) δ 5.85 (tm, J=110 Hz, 1H), 4.48-3.48 (m, 37.1H), 2.60-2.42 (m, 0.68H), 2.34-2.18 (m, 0.64H). 19F NMR (282 MHz, CDCl₃): −61.4-−63.9 (m, 33.58F), −126.2-−128.2 (m, 2F), −196.0-−198.9 (m, 0.29F). Density measured at 18° C. is 1.56 g/mL. Optical characterization: refractive index measured using a Schmidt Haensch ATR L Dispersion Refractometer at 20° C.: 1.3542 at 593.0 nm.

Compound L5 is an example the low index fluid including a modified polyether material, represented by formula (L5). Compound L5 is formed following a similar reaction scheme to compound L4 described above. Compound L5 is represented by formula L5 shown below.

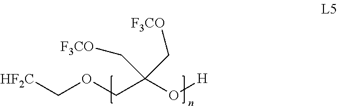

Compound L6 is an example the low index fluid including a modified polyether material, represented by formula (L6). Compound L6 is formed by reacting 2-(pentafluorosulfanyl)ethan-1-ol (reagent R5) with 2-((2-(pentafluoro-λ⁶-sulfaneyl)ethoxy)methyl)oxirane (precursor P6). The example reaction scheme is shown below:

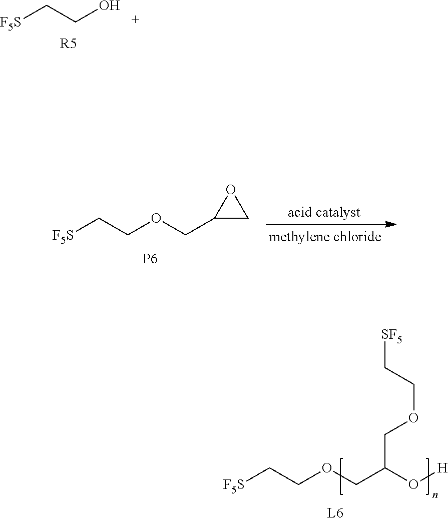

The reaction to form compound L6 includes combining anhydrous methylene chloride (6 mL) and an acid catalyst (0.067 g) in a 25 mL flask under an inert atmosphere of nitrogen. The mixture was cooled in an ice bath and then 2-(pentafluorosulfanyl)ethan-1-ol (reagent R5, 0.453 g) was added. 2-((2-(pentafluoro-λ⁶-sulfaneyl)ethoxy)methyl)oxirane (precursor P6, 6.06 g) was added dropwise over about 3 h. The mixture was stirred for an additional 60 min and then filtered through silica gel eluting with methylene chloride and ethyl acetate (60:40 v:v). Solvents were evaporated from the product mixture on a rotary evaporator and then a high vacuum line at 0.5 torr for about 1 h, while heating the product at 85° C. The residue was filtered through a 0.4 μm PTFE syringe filter to yield compound L6 as a clear and colorless viscous liquid (5.81 g, 89%). Chemical characterization of L6: ¹H NMR (300 MHz, CDCl₃) δ 4.20-3.16 (m). ¹⁹F NMR (282 MHZ, CDCl₃): δ 85.3-82.4 (m, 1F), 66.2-64.6 (m, 4F). Density measured at 18° C. is 1.613 g/mL. Optical characterization: refractive index measured using a Schmidt Haensch ATR L Dispersion Refractometer at 20° C.: 1.4154 at 593.0 nm.

Compound L7 is an example the low index fluid including a modified polyether material, represented by formula (L7). Compound L7 is formed by reacting 2-(trifluoromethoxy)ethanol (reagent 7) and tetraethylene glycol dimethyl ether (tetraglyme) with hexafluorobenzene (reagent R11). The example reaction scheme is shown below:

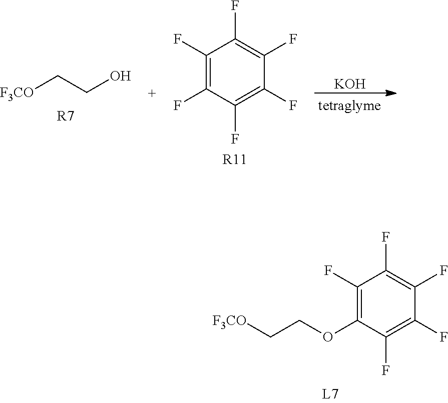

The reaction to form compound L7 includes charging 2-(trifluoromethoxy)ethanol (reagent 7, 17.4 g), tetraethylene glycol dimethyl ether (150 mL), hexafluorobenzene (reagent 11, 51.6 g), and potassium hydroxide (7.9 g) were charged into a 500 mL 3-neck flask under an inert atmosphere of nitrogen. The flask was heated to 75° C. for about 2 h. The reaction contents were diluted with methylene chloride (100 mL) and then washed with water (250 mL) twice, and saturated aqueous sodium chloride (200 mL). The organic mixture was stirred with anhydrous magnesium sulfate for about 1 h and then filtered. The crude product was then subjected to distillation using a glass Vigreux distillation column. The vapors collected with a boiling point of about 30° C. to 35° C./1 torr were collected as high purity compound L7 (33.5 g, 82%). Chemical characterization of L7: ¹H NMR (300 MHz, CDCl₃) δ 4.43-4.34 (m, 2H), 4.32-4.22 (m, 2H). ¹⁹F NMR (282 MHZ, CDCl₃): δ-61.8 (s, 3F), −157.1 (dm, J=19.5 Hz, 2F), −162.8 (tt, J=21.8, 4.0 Hz, 1F), −163.4 (tm, J=22.8 Hz, 2F). Density measured at 20° C. is 1.569 g/mL. Optical characterization: refractive index measured using a Schmidt Haensch ATR L Dispersion Refractometer at 20° C.: 1.3867 at 589.0 nm.

The brackets in the below formulas represent repeating units. For example, there may be n repeating units of the structure inside the corresponding parenthesis. m ranges from 0 to 400, p ranges from 0 to 400, and a sum of m and p range from 1 to 400. It is noted that m and p represent an average and the polysiloxane material may have a distribution of m or p around the average m or p. The modified polysiloxane material may have a relatively low refractive index and a relatively high density.

In some embodiments, the low index fluids may include the modified polysiloxane material represented by formula (III).

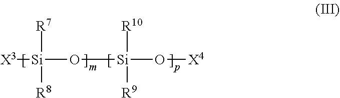

In certain embodiments, R⁷, R⁸, R⁹, and R¹⁰ are independently hydrogen, R⁵, or R⁶. R⁵ is an aromatic group represented by formula (II) above. R⁶ is a C₁-C₈ substituted hydrocarbyl wherein the hydrocarbyl is substituted with one or more fluorine, a fluoromethyl group, a difluoromethyl group, a trifluoromethoxy group, or a pentafluorosulfanyl group. For example, R⁶ may include (CH₂)_{w}CF₂H, (CH₂)_{w}O(CH₂)_{w}′CF₂H, (CH₂)_{w}CFH₂, (CH₂)_{w}O(CH₂) w CFH₂, (CH₂)_{w}O(CH₂)_{w}′SF₅, (CH₂)_{w}SF₅, (CH₂)_{w}OCF₃, (CH₂)_{w}O(CH₂)_{w}′OCF₃, or (CH₂)_{w}CH₃, (CH₂)_{w}O(CH₂)_{w}′CH₃ where w ranges from 0 to 8 and w′ ranges from 0 to 8.

In some embodiments, R⁷, R⁸, R⁹, and R¹⁰ of formula III are independently hydrogen, a methyl group, a difluoromethyl group, a 1,3-bis(trifluoromethoxy)-223-propane group, a 1,1,3,3-tetrafluoro-223-propane group, a (trifluoromethoxy)-423-benzene group, a 1,3-bis (trifluoromethoxy)-523-benzene group, a (trifluoromethoxy)-223-ethane group, a 1-fluoro-2-(trifluoromethoxy)-123-ethane group, a 1,3-difluoro-2-(trifluoromethoxy)-523-benzene group, a pentafluorosulfanyl methyl group, a pentafluorosulfanyl ethyl group, or a pentafluorosulfanyl propyl group. X³ and X⁴ are independently hydrogen, a hydroxyl group, ethylene, R⁶, O—R⁶, or Si(R¹¹R¹²R¹³). R¹¹, R¹², and R¹³ may include R⁵, R⁶, or one or more additional groups.

Compounds L8-L15, represented by formulas L8-L15 below, are examples of the low index fluid modified polysiloxane materials. X³ and X⁴ are independently hydrogen, a hydroxyl group, ethylene, R⁶, O—R⁶, or Si(R¹¹R¹²R¹³). R¹¹, R¹², R¹³ may independently include R⁵, R⁶, or one or more additional groups. R⁵ is an aromatic group represented by formula (II) above. R⁶ is a C₁-C₈ substituted hydrocarbyl wherein the hydrocarbyl is substituted with one or more fluorine, a fluoromethyl group, a difluoromethyl group, a trifluoromethoxy group, or a pentafluorosulfanyl group. For example, R⁶ may include (CH₂)_{w}CF₂H, (CH₂)_{w}O(CH₂)_{w}′CF₂H, (CH₂)_{w}CFH₂, (CH₂)_{w}O(CH₂)_{w}′CFH₂, (CH₂)_{w}O(CH₂)_{w}′SF₅, (CH₂)_{w}SF₅, (CH₂)_{w}OCF₃, or (CH₂)_{w}O(CH₂)_{w}′OCF₃, or (CH₂)_{w}CH₃, (CH₂)_{w}O(CH₂)_{w}′CH₃ where w ranges from 0 to 8 and w′ ranges from 0 to 8.

The brackets in the below formulas L8-L15 represent repeating units. For example, there may be n repeating units of the structure inside the corresponding parenthesis. m ranges from 0 to 400, p ranges from 0 to 400, and the sum of m and p range from 1 to 400. It is noted that m and p represent an average and the polysiloxane material may have a distribution of m or p around the average m or p. The modified polysiloxane material may have a relatively low refractive index and a relatively high density.

Compound L8 is an example the low index fluid including a modified polysiloxane material, represented by formula (L8).

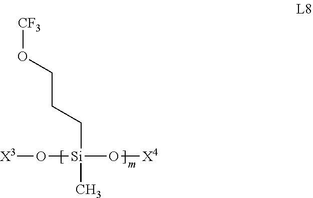

Compound L9 is an example the low index fluid including a modified polysiloxane material, represented by formula (L9).

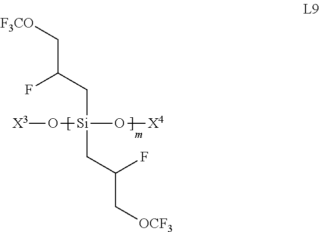

Compound L10 is an example the low index fluid including a modified polysiloxane material, represented by formula (L10).

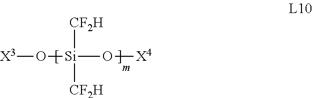

Compound L11 is an example the low index fluid including a modified polysiloxane material, represented by formula (L11).

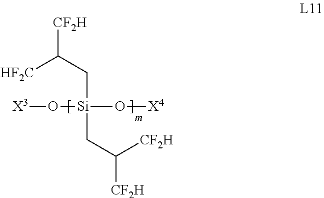

Compound L12 is an example the low index fluid including a modified polysiloxane material, represented by formula (L12).

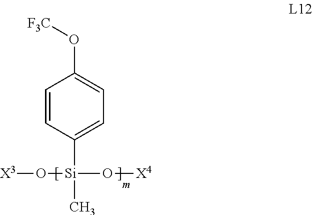

Compound L13 is an example the low index fluid including a modified polysiloxane material, represented by formula (L13).

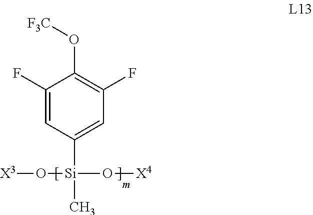

Compound L14 is an example the low index fluid including a modified polysiloxane material, represented by formula (L14).

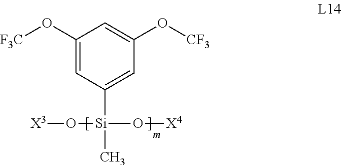

Compound L15 is an example the low index fluid including a modified polysiloxane material, represented by formula (L15).

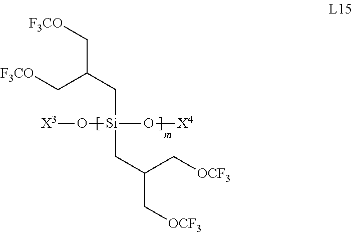

In some embodiments, the high index fluids may include the modified polysulfide materials. The modified polysulfide materials may include linear polymers and/or branched polymers. The modified polysulfide materials may have a high refractive index and a low density relative to the low index fluids. An example of the high index fluid modified polysulfide material is represented by formula (IV). The modified polysulfide material represented by formula (IV) is a linear polymer.

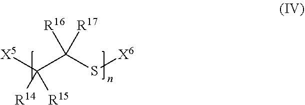

In some embodiments, the high index fluids may include the modified polysulfide material represented by formula (V). The modified polysulfide material represented by formula (V) is a linear polymer.

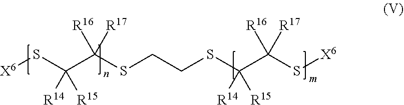

In some embodiments, the high index fluids may include the modified polysulfide material represented by formula (VI). The modified polysulfide material represented by formula (VI) is a branched polymer.

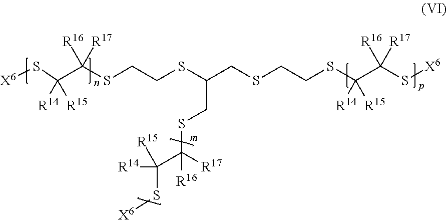

In some embodiments, the high index fluids may include the modified polysulfide material represented by formula (VII). The modified polysulfide material represented by formula (VII) is a branched polymer.

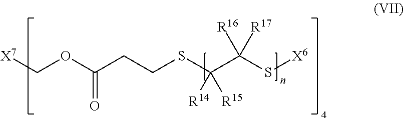

In certain embodiments, R¹⁴, R¹⁵, R¹⁶, and R¹⁷ of the modified polysulfide materials represented by formulas (IV-VII) are independently hydrogen, a branched saturated C₁-C₁₂ hydrocarbyl, (CH₂)_{Y}—H, (CH₂)_{Y}—O—(CH₂)_{Y}′-H, (CH₂)_{Y}—S—(CH₂)_{Y}′-H, CH₂—O—R¹⁸, and CH₂—S—R¹⁸. Y and Y¹ range from 1 to 20. R¹⁸ is an aromatic group represented by formula (VIII) or a thiophenyl group represented by formula (IX).

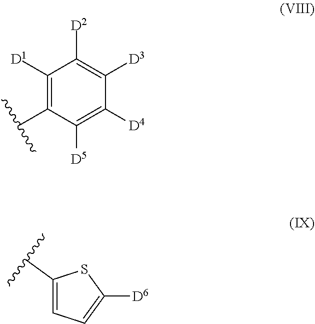

wherein D¹⁻⁶ is independently hydrogen, a branched saturated C₁-C₁₂ hydrocarbyl, (CH₂)_{Y}—H, (CH₂)_{Y}—O—(CH₂)_{Y}′-H, (CH₂)_{Y}—S—(CH₂)_{Y}—H, where Y and Y¹ range from 1 to 20.

X⁵ is R¹⁹, SR¹⁹, SR¹⁸, S(CH₂)_{Y}—H, S(CH₂)_{Y}—O—(CH₂)_{Y}′-OH, SCH₂CH₂COOR¹⁹, SCH₂CH₂CON(H) R¹⁹. X⁶ is hydrogen, R¹⁸, R¹⁹, (CH₂)_{Y}—H, (CH₂)_{Y}—O—(CH₂)_{Y}′-H, (CH₂)_{Y}—S—(CH₂)_{Y}′-H, (CH₂)_{Y}—OH, (CH₂)_{Y}—O—(CH₂)_{Y}′-OH, CH₂CH₂COOR¹⁹, CH₂CH₂CON(H) R¹⁹, where R¹⁹ is a branched saturated C₁-C₂₀ hydrocarbyl. X⁷ is C or Si

Compound H1 is an example the high index fluid including a modified polysulfide material, represented by formula (H1). Compound H1 is formed by reacting 1-butanethiol (reagent R12) and propylene sulfide (reagent R13). The example reaction scheme is shown below:

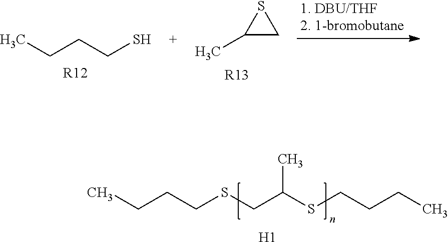

The reaction to form compound H1 includes adding tetrahydrofuran (THF, 300 mL) to a 1 L 3-neck flask under an inert atmosphere of nitrogen. 1-butanethiol (reagent R12, 13.29 g) and 1,8-diazabicyclo[5.4.0]undec-7-ene (DBU, 24.7 g) were then added to the flask. The flask is heated to an internal temperature of 50° C., and then propylene sulfide (reagent R13, 120.1 g) was added dropwise over about 40 minutes. The reaction was allowed to stir for about 1.5 h at 50° C., then 1-bromobutane (30.1 g) was added. The reaction was stirred at 50° C. for about 16 h and then cooled. Tetrahydrofuran was evaporated on a rotary evaporator and then the crude product was dissolved in methylene chloride (300 mL). The organic solution was washed with 5% aqueous hydrochloric acid (150 mL) three times, water (150 mL), saturated aqueous sodium bicarbonate (150 mL), and saturated aqueous sodium chloride (150 mL). The organic solution was then stirred with anhydrous magnesium sulfate (15 g) for about 14 h. Solvents were evaporated from the product mixture on a rotary evaporator and then a high vacuum line at 0.5 torr for about 2 h, while heating the product at 110° C. The residue was filtered through a 0.05 μm filter to yield compound H1 (112 g) as a clear and colorless viscous liquid. Chemical characterization: 1H NMR (300 MHZ, CDCl₃) δ 3.2-2.3 (m, 40.36H), 1.7-1.1 (m, 45.27H), 0.91 (t, J=7.3 Hz, 6H). Density measured at 20° C. is 1.094 g/mL. Optical characterization: refractive index measured using a Schmidt Haensch ATR L Dispersion Refractometer at 20° C.: 1.5708 at 593.0 nm.

Compound H2 is an example the high index fluid including a modified polysulfide material, represented by formula (H2). Compound H2 is represented by formula H2 shown below.

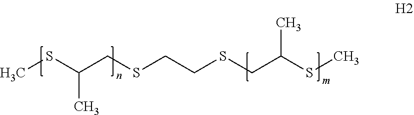

Compound H3 is an example the high index fluid including a modified polysulfide material, represented by formula (H3). Compound H3 is formed by reacting 1,2-ethanedithiol (reagent R14) and propylene sulfide (reagent R13). The example reaction scheme is shown below:

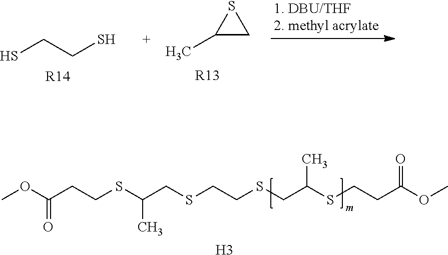

The reaction to form compound H3 includes adding anhydrous tetrahydrofuran (30 mL) was added to a 100 mL 3-neck flask under an inert atmosphere of nitrogen. 1,2-ethanedithiol (reagent R14, 1.20 g) and 1,8-diazabicyclo[5.4.0]undec-7-ene (DBU, 0.381 g) were then added to the flask. The flask was heated to an internal temperature of 60° C., and then propylene sulfide (reagent R13, 9.49 g) was added slowly over about 15 minutes. The reaction was allowed to stir for about 90 min at 60° C. The reaction was cooled to 35° C., then methyl acrylate (2.76 g) was added by syringe. After stirring at room temperature overnight, tetrahydrofuran was evaporated on a rotary evaporator and then the crude product was dissolved in methylene chloride (75 mL). The organic solution was washed with 5% aqueous hydrochloric acid (50 mL) three times, water (50 mL), saturated aqueous sodium bicarbonate (50 mL), and saturated aqueous sodium chloride (50 mL). The organic solution was then dried with anhydrous magnesium sulfate. Solvents were evaporated from the product mixture on a rotary evaporator and then a high vacuum line at 0.5 torr for about 2 h, while heating the product at 80° C. The residue was filtered through a 0.4 μm filter to yield compound H3 (12.1 g) as a clear and colorless viscous liquid. Chemical characterization: 1H NMR (61 MHz, CDCl₃) δ 3.68 (s, 6H), 3.30-2.24 (m, 39.74H), 1.34 (d, J=6.1 Hz, 28.51H). Density measured at 20° C. is 1.155 g/mL. Optical characterization: refractive index measured using a Schmidt Haensch ATR L Dispersion Refractometer at 20° C.: 1.5731 at 593.0 nm.

Compound H4 is an example the high index fluid including a modified polysulfide material, represented by formula (H4). Compound H4 is formed by reacting 4-(mercaptomethyl)-1,8-dimercapto-3,6-dithiaoctane (reagent R15) and propylene sulfide (reagent R13). The example reaction scheme is shown below:

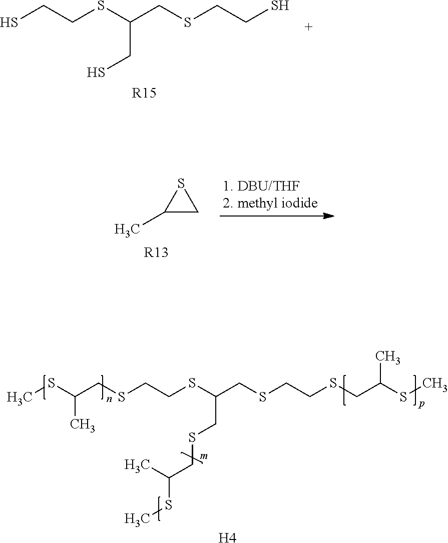

The reaction to form compound H4 includes adding anhydrous tetrahydrofuran (THF, 30 mL) to a 100 mL 3-neck flask under an inert atmosphere of nitrogen. 4-(mercaptomethyl)-1,8-dimercapto-3,6-dithiaoctane (reagent R15, 3.630 g) and 1,8-diazabicyclo[5.4.0]undec-7-ene (DBU, 6.5 g) were then added to the flask. The flask was heated to an internal temperature of 60° C., and then propylene sulfide (reagent R13, 10.0 g) was added dropwise over about 15 mins. The reaction was allowed to stir for about 45 min at 60° C. The reaction was cooled to room temperature, then methyl iodide (6.5 g) was added by syringe. After stirring at room temperature overnight, tetrahydrofuran was evaporated on a rotary evaporator and then the crude product was dissolved in methylene chloride (75 mL). The organic solution was washed with 5% aqueous hydrochloric acid (50 mL) three times, water (50 mL), saturated aqueous sodium bicarbonate (50 mL), and saturated aqueous sodium chloride (50 mL). The organic solution was then dried with anhydrous magnesium sulfate. Solvents were evaporated from the product mixture on a rotary evaporator and then a high vacuum line at 0.5 torr for about 2 h, while heating the product at 80° C. The residue was filtered through a 0.4 μm filter to yield compound H4 (13.8 g) as a clear and colorless viscous liquid. Chemical characterization: 1H NMR (61 MHz, CDCl₃) δ 3.30-2.32 (m, 44.02H), 2.11 (s, 9.00H), 1.35 (d, J=5.9 Hz, 30.22H). Density measured at 20° C. is 1.153 g/mL. Optical characterization: refractive index measured using a Schmidt Haensch ATR L Dispersion Refractometer at 20° C.: 1.5955 at 593.0 nm.

Compound H5 is an example the high index fluid including a modified polysulfide material, represented by formula (H5). Compound H5 is represented by formula H5 shown below.

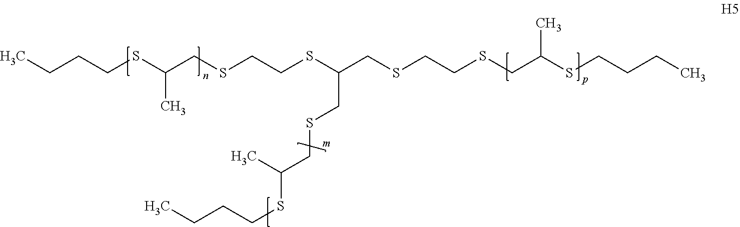

Compound H6 is an example the high index fluid including a modified polysulfide material, represented by formula (H6). Compound H6 is represented by formula H6 shown below.

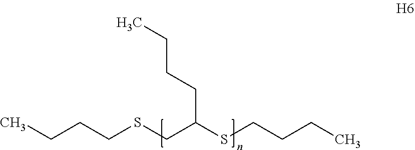

Compound H7 is an example the high index fluid including a modified polysulfide material, represented by formula (H7). Compound H7 is formed by reacting 1,2-ethanedithiol (reagent R14) and 1,2-epithiodecane (reagent R16). The example reaction scheme is shown below:

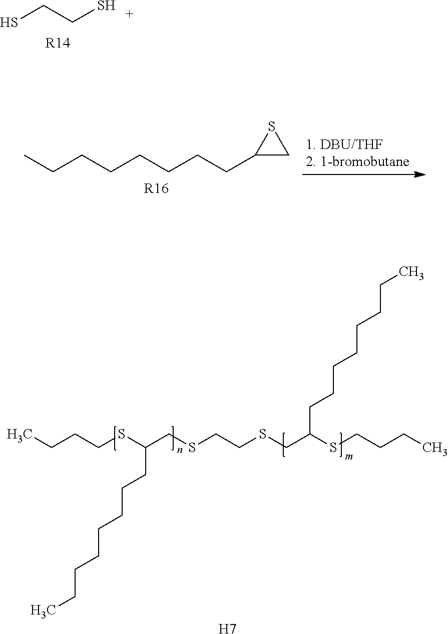

The reaction to form compound H7 includes adding anhydrous tetrahydrofuran (THF, 40 mL) to a 250 mL 3-neck flask under an inert atmosphere of nitrogen. 1,2-ethanedithiol (reagent R14, 1.109 g), 1,8-diazabicyclo[5.4.0]undec-7-ene (DBU, 3.812 g) and 1,2-epithiodecane (reagent R16, 20.0 g) were then added to the flask at room temperature. The reaction was heated to reflux overnight. After cooling the reaction to 50° C., 1-bromobutane (3.95 g) was injected by syringe. The reaction maintained at 50° C. for about 2 h. Tetrahydrofuran was evaporated on a rotary evaporator, the crude product dissolved in methylene chloride (150 mL) and washed with 5% aqueous hydrochloric acid (50 mL) three times, water (50 mL), saturated aqueous sodium bicarbonate (50 mL), and saturated aqueous sodium chloride (50 mL). The organic solution was then dried with anhydrous magnesium sulfate. Solvents were evaporated from the product mixture on a rotary evaporator and then a high vacuum line at 0.5 torr for about 2 h, while heating the product at 80° C. The residue was filtered through a 0.4 μm filter to yield compound H7 (19.5 g) as a clear and colorless viscous liquid. Chemical characterization: 1H NMR (300z MHz, CDCl₃) § 3.15-2.35 (m, 44.19H), 2.04-1.64 (m, 11.71H), 1.63-1.00 (m, 161.86H), 1.00-0.60 (m, 42.19H). Density measured at 20° C. is 0.950 g/mL. Optical characterization: refractive index measured using a Schmidt Haensch ATR L Dispersion Refractometer at 20° C.: 1.5119 at 593.0 nm.

Compound H8 is an example the high index fluid including a modified polysulfide material, represented by formula (H8). Compound H8 is represented by formula H8 shown below.

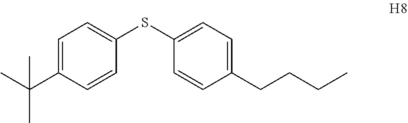 [SMILES: CCCCc1ccc(Sc2ccc(C(C)(C)C)cc2)cc1]

The refractive indices and densities of the low index fluids (compounds L1-L15) and high index fluids (compounds H1-H8) are provided in Table 1. It should be noted, the refractive indices of the modified polyether materials (compounds L1-L7) and the modified polysulfide materials (compounds H1-H8) are measured using a refractometer at 20° C. at 593.0 nm and a kinematic viscometer at 20° C., respectively. The refractive indices of the modified polysiloxane materials (compounds L8-L15) are calculated using ACD Labs Percepta Software package.

TABLE 1
Refractive indices (RI) and densities of the low index fluids
(compounds L1-L15) and high index fluids (compounds H1-H8).

| | L1 | L2 | L3 | L4 | L5 | L6 | L7 |
|---|---|---|---|---|---|---|---|
| RI | 1.37 | 1.40 | 1.47 | 1.36 | 1.35 | 1.44 | 1.39 |
| Density (g/mL) | 1.46 | 1.45 | 1.59 | 1.56 | 1.56 | 1.52 | 1.57 |

| | L8 | L9 | L10 | L11 | L12 | L13 | L14 | L15 |
|---|---|---|---|---|---|---|---|---|
| Calculated RI | 1.40 | 1.37 | 1.33 | 1.34 | 1.51 | 1.49 | 1.47 | 1.37 |
| Calculated | 1.24 | 1.41 | 1.48 | 1.42 | 1.41 | 1.52 | 1.58 | 1.46 |
| Density (g/mL) | | | | | | | | |
| | H1 | H2 | H3 | H4 | H5 | H6 | H7 | H8 |
| RI | 1.57 | 1.59 | 1.57 | 1.59 | 1.58 | 1.53 | 1.51 | 1.57 |
| Density (g/mL) | 1.09 | 1.13 | 1.15 | 1.15 | 1.10 | 1.06 | 0.95 | 1.00 |

The refractive index of compounds L1-L7 may range from about 1.30 to 1.50, from about 1.35 to 1.49, from about 1.35 to 1.47, from about 1.36 to 1.44, from about 1.39 to about 1.47, at least 1.30, at least 1.35, at least 1.40, less than 1.50, about 1.33, about 1.39, about 1.44, about 1.47, and the like. The density of compounds L1-L7 may range from about 1.40 to 1.65 g/mL, from about 1.45 to 1.59 g/mL, from about 1.46 to 1.59 g/mL, at least 1.40 g/mL, at least 1.45 g/mL, at least 1.47 g/mL, less than 1.80 g/mL, less than 1.60 g/mL, about 1.52 g/mL, about 1.45 g/mL, about 1.56 g/mL, about 1.57 g/mL, and the like.

The refractive index of compounds L8-L15 may range from about 1.30 to 1.55, from about 1.33 to 1.51, from about 1.33 to 1.47, from about 1.33 to 1.46, at least 1.30, at least 1.35, at least 1.40, less than 1.50, about 1.33, about 1.34, about 1.40, about 1.51, and the like. The density of compounds L8-L15 may range from about 1.20 to 1.60 g/mL, from about 1.24 to 1.52 g/mL, from about 1.41 to 1.58 g/mL, at least 1.20 g/mL, at least 1.30 g/mL, at least 1.40 g/mL, less than 1.80 g/mL, less than 1.60 g/mL, about 1.52 g/mL, about 1.58 g/mL, about 1.46 g/mL, about 1.41 g/mL, and the like.

The refractive index of compounds H1-H8 may range from about 1.50 to 1.65, from about 1.51 to 1.59, from about 1.53 to 1.59, from about 1.57 to 1.59, at least 1.40, at least 1.45, at least 1.50, more than 1.30, about 1.51, about 1.53, about 1.58, about 1.59, and the like. The density of compounds H1-H8 may range from about 0.90 to 1.25 g/mL, from about 0.95 to 1.20 g/mL, from about 1.00 to 1.15 g/mL, at least 0.90 g/mL, at least 0.95 g/mL, less than 1.35 g/mL, less than 1.30 g/mL, about 1.00 g/mL, about 1.10 g/mL, about 1.13 g/mL, about 1.15 g/mL, and the like.

In certain embodiments, the first and second optical fluids 92-1, 92-2 of FIG. 7 may be materials with properties that are selected such that the first and second fluid-filled chambers 94-1, 94-2 mutually compensate one another for gravity sag. The first optical fluid 92-1 may be selected from compounds L1-L15. The second optical fluid 92-2 may be selected from compounds H1-H8.

The difference in refractive indices (ARI) and the difference in densities (Ap) of a portion of the low index fluids, the modified polyether materials (compounds L1-L7), and a portion of the high index fluids, the modified polysulfide materials (compounds H1-H8), are provided in Table 2.

TABLE 2
Difference in refractive indices (ΔRI) and the
difference in densities (Δρ) between the modified
polysulfide materials (compounds H1-H8) and the modified
polyether materials (compounds L1-L7)

| | | H1 | H2 | H3 | H4 | H5 | H6 | H7 | H8 |
|---|---|---|---|---|---|---|---|---|---|
| ΔRI | L1 | 0.20 | 0.22 | 0.20 | 0.22 | 0.21 | 0.16 | 0.14 | 0.20 |
| Δρ | | 0.37 | 0.33 | 0.31 | 0.31 | 0.36 | 0.40 | 0.51 | 0.46 |
| ΔRI | L2 | 0.17 | 0.19 | 0.17 | 0.19 | 0.18 | 0.13 | 0.11 | 0.17 |
| Δρ | | 0.36 | 0.32 | 0.30 | 0.30 | 0.35 | 0.39 | 0.50 | 0.45 |
| ΔRI | L3 | 0.10 | 0.12 | 0.10 | 0.12 | 0.11 | 0.06 | 0.04 | 0.10 |
| Δρ | | 0.50 | 0.46 | 0.44 | 0.44 | 0.49 | 0.53 | 0.64 | 0.59 |
| ΔRI | L4 | 0.21 | 0.23 | 0.21 | 0.23 | 0.22 | 0.17 | 0.15 | 0.21 |
| Δρ | | 0.47 | 0.43 | 0.41 | 0.41 | 0.46 | 0.50 | 0.61 | 0.56 |
| ΔRI | L5 | 0.22 | 0.24 | 0.22 | 0.24 | 0.23 | 0.18 | 0.16 | 0.22 |
| Δρ | | 0.47 | 0.43 | 0.41 | 0.41 | 0.46 | 0.50 | 0.61 | 0.56 |
| ΔRI | L6 | 0.13 | 0.15 | 0.13 | 0.15 | 0.14 | 0.09 | 0.07 | 0.13 |
| Δρ | | 0.43 | 0.39 | 0.37 | 0.37 | 0.42 | 0.46 | 0.57 | 0.52 |
| ΔRI | L7 | 0.18 | 0.20 | 0.18 | 0.20 | 0.19 | 0.14 | 0.12 | 0.18 |
| Δρ | | 0.48 | 0.44 | 0.42 | 0.42 | 0.47 | 0.51 | 0.62 | 0.57 |

The difference in refractive index between the modified polysulfide materials (compounds H1-H8) and the modified polyether materials (compounds L1-L7), may range from about 0.04 to 0.24, about 0.10 to 0.24, about 0.15 to 0.24, about 0.16 to 0.22, at least 0.10, at least 0.15, at least 0.20, less than 0.50, less than 0.40, less than 0.30, etc. The difference in density between the modified polysulfide materials (compounds H1-H8) and the modified polyether materials (compounds L1-L7) may range from about 0.30 to 0.65, about 0.42 to 0.65, about 0.50 to 0.65, about 0.55 to 0.65, about 0.55 to 0.61, at least 0.30 g/cm³, at least 0.50 g/cm³, at least 0.70 g/cm³, less than 1.50 g/cm³, less than 1.30 g/cm³, less than 1.10 g/cm³, less than 1.00 g/cm³.

In some embodiments, the first and second optical fluids 92-1, 92-2 may be selected from the modified polyether materials (represented by formula I), modified polysiloxane materials (represented by formula III), modified polyester materials, and modified polysulfide materials (represented by formulas IV-VII). Fluid-filled chambers of the lens modules may include optical fluids to compensate one another to mitigate optical aberrations otherwise caused by gravity effects. As such, the first and second optical fluids 92-1 and 92-2 may be materials with properties that are selected such that that the first and second fluid-filled chambers 94-1, 94-2 mutually compensate one another for gravity induced aberrations. For example, compound L4 and compound H8 may be selected as the first and second optical fluids 92-1, 92-2, respectively. In another example, compound L5 and compound H8 may be selected as the first and second optical fluids 92-1, 92-2, respectively. Additionally and/or alternatively, compound L5 and compound H7 may be selected as the first and second optical fluids 92-1, 92-2, respectively. Without wishing to be bound by theory, gravity sag may be mitigated by selecting a pair of the first and second optical fluids 92-1, 92-2 with a relatively high difference in refractive index and density. The first and second optical fluids 92-1, 92-2 may reduce light beam deflection to improve performance of lens elements of the lens modules of electronic devices. In this manner, gravity induced changes in curvature of the lens element may be achieved using PFAS-free optical fluid pairs.

Technical effects of the disclosed embodiments include systems and processes directed to PFAS-free optical fluids for use in lens modules of electronic devices. For example, a lens module of an electronic device may include two fluid-filled chambers with optical fluids having different properties to mitigate gravity induced aberrations/sagging. The optical fluids may be formed from PFAS-free materials such as modified polyether materials, modified polysiloxane materials, modified polyester materials, modified polysulfide materials, and modified polypropylene materials. The optical fluids presented herein include relatively low refractive index and relatively high-density optical oils (e.g., compounds L1-L15 of Table 1) and relatively high refractive index and relatively low-density optical oils (e.g., compounds H1-H8 of Table 1). Further, technical effects of the disclosed embodiments include synthesis of polyether-based low index optical fluids. Additionally, technical effects of the disclosed embodiments include pairing low index and the high index optical oils in the lens modules of electronic devices to mitigate gravity induced aberrations.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ,” it is intended that such elements are to be interpreted under 35 U.S.C. 112 (f). However, for any claims containing elements designated in any other manner, it is intended that such elements are not to be interpreted under 35 U.S.C. 112 (f).

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

1. A fluid represented by Formula (I) 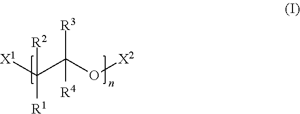
wherein:
R¹, R², R³, and R⁴ are independently hydrogen, a methyl group, a trifluoromethoxy methyl group, a difluoromethyl group, a 1-ethoxy-2,3,4,5,6-pentafluorophenyl, (2-ethoxyethyl) pentafluoro sulfane group, a pentafluorosulfanyl group, or a pentafluorosulfanyl methyl group;
X¹ and X² are independently hydrogen, fluorine, chlorine, bromine, a hydroxyl group, a methyl group, a methylpropane, a 2-trifluoromethoxy ethoxy group, a 2,2-difluoroethoxy group, a 2-(pentafluorosulfanyl)ethoxy group, a pentafluorophenoxy group, R⁵, O—R⁵, S—R⁵, R⁶, O—R⁶, or S—R⁶;
R⁵ is a substituted aromatic group;
R⁶is CH₂)_{w}CF₂H, (CH₂)_{w}O(CH₂)_{w}′CF₂H, (CH₂)_{w}CFH₂, (CH₂)_{w}O(CH₂)_{w}′CFH₂, (CH₂)_{w}O(CH₂)_{w}′SF₅, (CH₂)_{w}SF₅, (CH₂)_{w}OCF₃, or (CH₂)_{w}O(CH₂)_{w}′OCF₃, (CH₂)_{w}CH₃, or (CH₂)_{w}O(CH₂)_{w}′CH₃, wherein w ranges from 0 to 8 and w′ ranges from 0 to 8; and
n is 1 to 100.

2. The fluid of claim 1, wherein R¹, R², R³, and R⁴ are independently hydrogen or a trifluoromethoxy methyl group.

3. The fluid of claim 1, wherein X¹ and X² are independently hydrogen or a 2,2-difluoroethoxy group.

4. The fluid of claim 1, wherein R¹, R², R³, and R⁴ are independently hydrogen, a 1-ethoxy-2,3,4,5,6-pentafluorophenyl, or (2-ethoxyethyl) pentafluoro sulfane group.

5. The fluid of claim 1, wherein X¹ and X² are independently hydrogen, a 2-trifluoromethoxy ethoxy group, 2-(pentafluorosulfanyl)ethoxy group, or a pentafluorophenoxy group.

6. The fluid of claim 1, wherein the fluid has a density at 20° C. ranging from 1.20 to 1.85 g/mL.

7. The fluid of claim 1, wherein the fluid has a refractive index ranging from 1.30 to 1.55.

8. The fluid of claim 1, wherein the fluid is selected from the group represented by the following formulas: 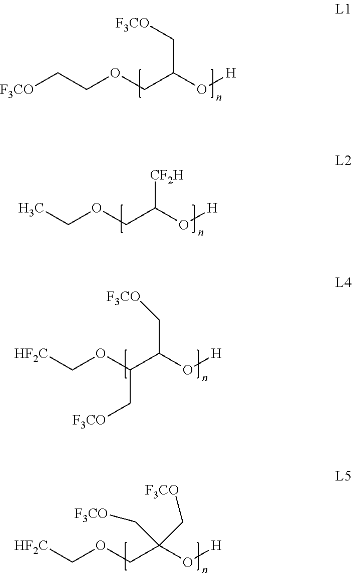
wherein n is 1 to 100.

9. The fluid of claim 1, wherein the fluid is selected from the group represented by the following formulas: 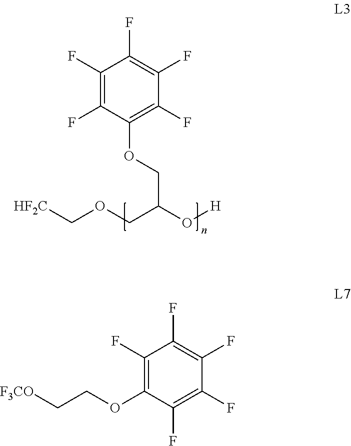
wherein n is 1 to 100.

10. The fluid of claim 1, wherein the fluid is compound L6, represented by formula L6: 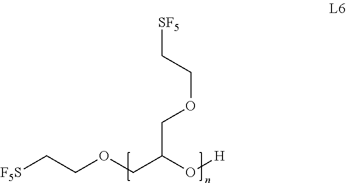
wherein n is 1 to 100.

11. A lens module, comprising:
a first fluid-filled chamber comprising a first optical fluid represented by Formula (I), 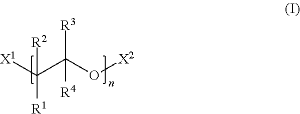
wherein:
R¹, R², R³, and R⁴ are independently hydrogen, a methyl group, a trifluoromethoxy methyl group, a difluoromethyl group, a 1-ethoxy-2,3,4,5,6-pentafluorophenyl, (2-ethoxyethyl) pentafluoro sulfane group, a pentafluorosulfanyl group, or a pentafluorosulfanyl methyl group;
X¹ and X² are independently hydrogen, fluorine, chlorine, bromine, a hydroxyl group, a methyl group, a methylpropane, a 2-trifluoromethoxy ethoxy group, a 2,2-difluoroethoxy group, a 2-(pentafluorosulfanyl)ethoxy group, or a pentafluorophenoxy group, R⁵, O—R⁵, S—R⁵, R⁶, O—R⁶, or S—R⁶;
R⁵ is a substituted aromatic group; and
R⁶is CH₂)_{w}CF₂H, (CH₂)_{w}O(CH₂)_{w}′CF₂H, (CH₂)_{w}CFH₂, (CH₂)_{w}O(CH₂)_{w}′CFH₂, (CH₂)_{w}O(CH₂)_{w}′SF₅, (CH₂)_{w}SF₅, (CH₂)_{w}OCF₃, or (CH₂)_{w}O(CH₂)_{w}′OCF₃, (CH₂)_{w}CH₃, or (CH₂)_{w}O(CH₂)_{w}′CH₃, wherein w ranges from 0 to 8 and w′ ranges from 0 to 8; and
n is 1 to 100; and
a second fluid-filled chamber comprising a second optical fluid selected from the group represented by the following formulas: 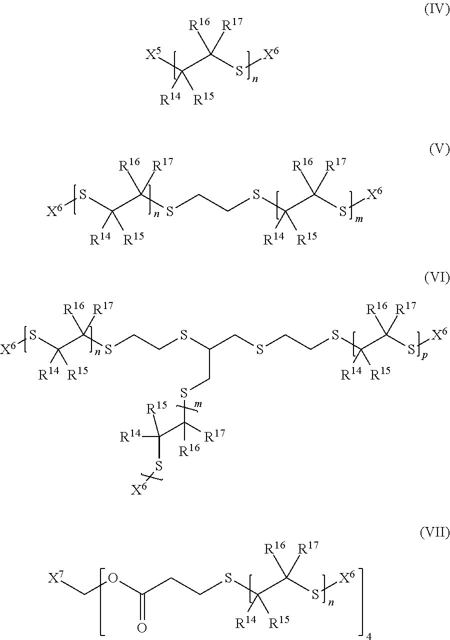
wherein:
R¹⁴, R¹⁵, R¹⁶, and R¹⁷ are independently hydrogen, a branched saturated C₁-C₁₂ hydrocarbyl, (CH₂)_{Y}—H, (CH₂)_{Y}—O—(CH₂)_{Y}—H, (CH₂)_{Y}—S—(CH₂)_{Y}′-H, CH₂—O—R¹⁸, or CH₂—S—R¹⁸, and wherein R¹⁸ is a functionalized aromatic group or a thiophenyl group;
X⁵ is R¹⁹, SR¹⁹, SR¹⁸, S(CH₂)_{Y}—H, S(CH₂)_{Y}—O—(CH₂)_{Y}′-OH, SCH₂CH₂COOR¹⁹, SCH₂CH₂CON(H) R¹⁹;
X⁶ is hydrogen, R¹⁸, R¹⁹, (CH₂)_{Y}—H, (CH₂)_{Y}—O—(CH₂)_{Y}′-H, (CH₂)_{Y}—S—(CH₂)_{Y}′-H, (CH₂)_{Y}—OH, (CH₂)_{Y}—O—(CH₂)_{Y}′-OH, CH₂CH₂COOR¹⁹, CH₂CH₂CON(H) R¹⁹, wherein R¹⁹ is a branched saturated C₁-C₂₀ hydrocarbyl;
X⁷ is C or Si;
Y and Y¹ range from 1 to 20; and
n, m, and p are independently 1 to 100.

12. The lens module of claim 11, wherein R¹, R², R³, and R⁴ are independently hydrogen or a trifluoromethoxy methyl group, and wherein X¹ and X² are independently hydrogen or a 2,2-difluoroethoxy group.

13. The lens module of claim 11, wherein the second optical fluid is selected from the group represented by the following formulas: 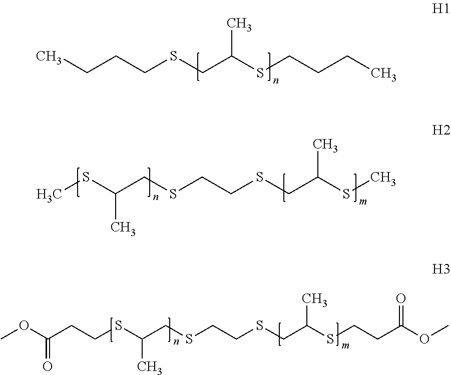
wherein n and m are independently 1 to 100.

14. The lens module of claim 11, wherein the second optical fluid is selected from the group represented by the following formulas: 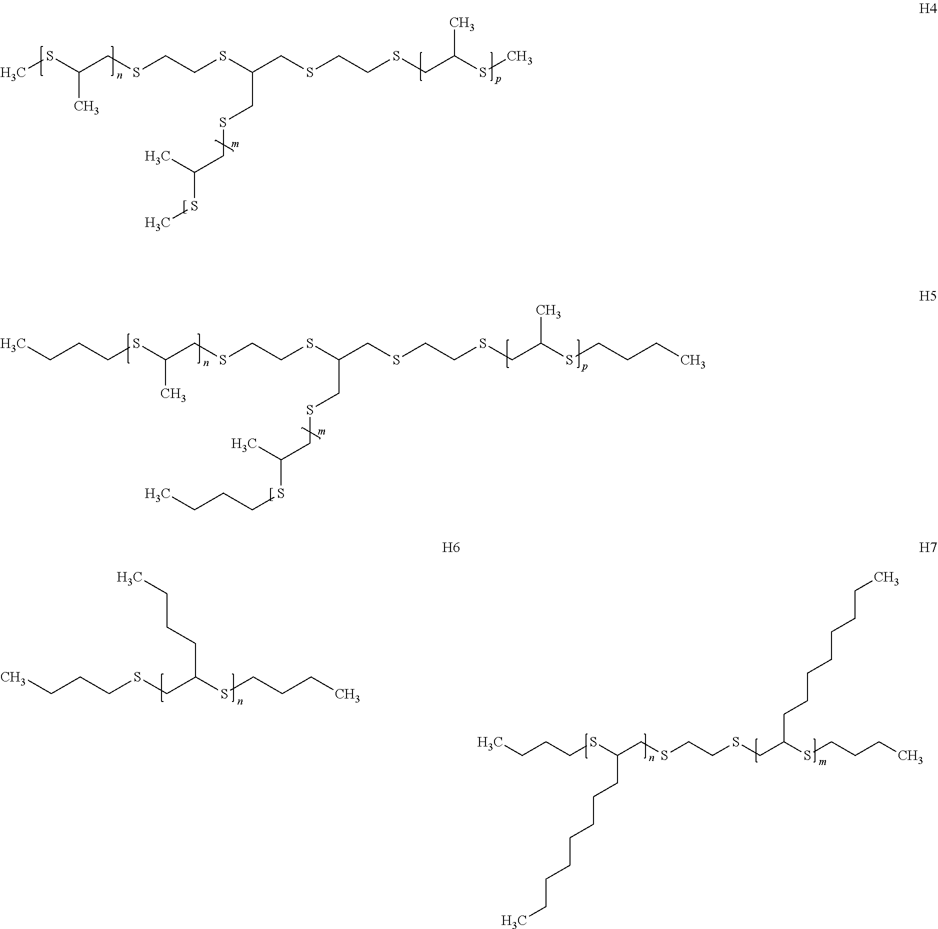
wherein n, m, and p are independently 1 to 100.

15. The lens module of claim 11, wherein the second optical fluid is compound H8, represented by formula H8:

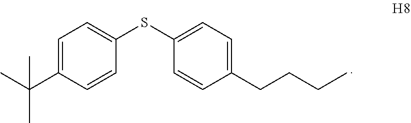 [SMILES: CCCCc1ccc(Sc2ccc(C(C)(C)C)cc2)cc1]

16. The lens module of claim 11, wherein the first optical fluid has a density at 20° C. ranging from 1.20 to 1.85 g/mL and a refractive index of the ranging from 1.30 to 1.55.

17. The lens module of claim 11, wherein the second optical fluid has a density at 20° C. ranging from 0.70 to 1.30 g/mL and a refractive index of the ranging from 1.50 to 1.75.

18. The lens module of claim 11, wherein both the first and second optical fluids comprise less than 50 ppm of any per- and polyfluoroalkyl substances (PFAS).

19. A lens module, comprising:
a first lens element;
a second lens element;
a third lens element, wherein the second lens element is interposed between the first and third lens elements;
a first optical fluid interposed between the first and second lens elements, wherein the first optical fluid has a density at 20° C. ranging from 1.20 to 1.85 g/mL and a refractive index of the ranging from 1.30 to 1.55; and
a second optical fluid interposed between the second and third lens elements, wherein the second optical fluid has a density at 20° C. ranging from 0.70 to 1.30 g/mL and a refractive index of the ranging from 1.50 to 1.75;
wherein both the first and second optical fluids comprise less than 50 ppm of any per- and polyfluoroalkyl substances (PFAS).

20. The lens module of claim 19, wherein the first optical fluid is a modified polyether material or a polysiloxane material and wherein the second optical fluid is a modified polysulfide material.

21. A fluid represented by Formula (III) 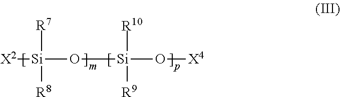
wherein:
R⁷, R⁸, R⁹, and R¹⁰ are independently hydrogen, a methyl group, a difluoromethyl group, a 1,3-bis(trifluoromethoxy)-223-propane group, a 1,1,3,3-tetrafluoro-223-propane group, a (trifluoromethoxy)-423-benzene group, a 1,3-bis (trifluoromethoxy)-523-benzene group, a (trifluoromethoxy)-223-ethane group, a 1-fluoro-2-(trifluoromethoxy)-123-ethane group, or a 1,3-difluoro-2-(trifluoromethoxy)-523-benzene group, a pentafluorosulfanyl methyl group, a pentafluorosulfanyl ethyl group, or a pentafluorosulfanyl propyl group;
X³ and X⁴ are independently hydrogen, a hydroxyl group, ethylene, R⁶, O—R⁶, or Si(R¹¹R¹²R¹³), wherein R¹¹, R¹², and R¹³ are independently an aromatic group or a C₁-C₈ substituted hydrocarbyl wherein the hydrocarbyl is substituted with one or more fluorine, a fluoromethyl group, a difluoromethyl group, a trifluoromethoxy group, or a pentafluorosulfanyl group; and
m and p are independently 0 to 100, wherein a sum of m and p ranges from 1 to 200.

22. A fluid represented by Formula (IV) 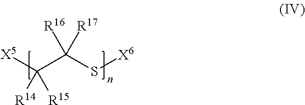
wherein:
R¹⁴, R¹⁵, R¹⁶, and R¹⁷ are independently hydrogen, a branched saturated C₁-C₁₂ hydrocarbyl, (CH₂)_{Y}—H, (CH₂)_{Y}—O—(CH₂)_{Y}′-H, (CH₂)_{Y}—S—(CH₂)_{Y}′-H, CH₂—O—R¹⁸, or CH₂—S—R¹⁸, and wherein R¹⁸ is a functionalized aromatic group or a thiophenyl group;
X⁵ is R¹⁹, SR¹⁹, SR¹⁸, S(CH₂)_{Y}—H, S(CH₂)_{Y}—O—(CH₂)_{Y}′-OH, SCH₂CH₂COOR¹⁹, SCH₂CH₂CON(H) R¹⁹;
X⁶ is hydrogen, R¹⁸, R¹⁹, (CH₂)_{Y}—H, (CH₂)_{Y}—O—(CH₂)_{Y}′-H, (CH₂)_{Y}—S—(CH₂)_{Y}′-H, (CH₂)_{Y}—OH, (CH₂)_{Y}—O—(CH₂)_{Y}′-OH, CH₂CH₂COOR¹⁹, CH₂CH₂CON(H) R¹⁹, wherein R¹⁹ is a branched saturated C₁-C₂₀ hydrocarbyl; Y and Y¹ range from 1 to 20; and
n is 1 to 100.

23. A fluid represented by Formula (V) 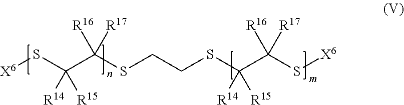
wherein:
R¹⁴, R¹⁵, R¹⁶, and R¹⁷ are independently hydrogen, a branched saturated C₁-C₁₂ hydrocarbyl, (CH₂)_{Y}—H, (CH₂)_{Y}—O—(CH₂)_{Y}′-H, (CH₂)_{Y}—S—(CH₂)_{Y}′-H, CH₂—O—R¹⁸, or CH₂—S—R¹⁸, and wherein R¹⁸ is a functionalized aromatic group or a thiophenyl group;X⁶ is hydrogen, R¹⁸, R¹⁹, (CH₂)_{Y}—H, (CH₂)_{Y}—O—(CH₂)_{Y}′-H, (CH₂)_{Y}—S—(CH₂)_{Y}′-H, (CH₂)_{Y}—OH, (CH₂)_{Y}—O—(CH₂)_{Y}′-OH, CH₂CH₂COOR¹⁹, CH₂CH₂CON(H) R¹⁹, wherein R¹⁹ is a branched saturated C₁-C₂₀ hydrocarbyl;
Y and Y¹ range from 1 to 20; and
n and m are independently 1 to 100.

24. A fluid represented by Formula (VI) 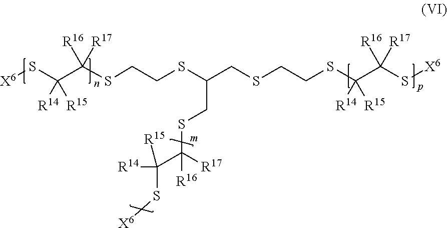
wherein:
R¹⁴, R¹⁵, R¹⁶, and R¹⁷ are independently hydrogen, a branched saturated C₁-C₁₂ hydrocarbyl, (CH₂)_{Y}—H, (CH₂)_{Y}—O—(CH₂)_{Y}′-H, (CH₂)_{Y}—S—(CH₂)_{Y}—H, CH₂—O—R¹⁸, or CH₂—S—R¹⁸, and wherein R¹⁸ is a functionalized aromatic group or a thiophenyl group;X⁶ is hydrogen, R¹⁸, R¹⁹, (CH₂)_{Y}—H, (CH₂)_{Y}—O—(CH₂)_{Y}′-H, (CH₂)_{Y}—S—(CH₂)_{Y}—H, (CH₂)_{Y}—OH, (CH₂)_{Y}—O—(CH₂)_{Y}′-OH, CH₂CH₂COOR¹⁹, CH₂CH₂CON(H) R¹⁹, wherein R¹⁹ is a branched saturated C₁-C₂₀ hydrocarbyl;
Y and Y¹ range from 1 to 20; and
n, m, and p are independently 1 to 100.

25. A fluid represented by Formula (IV) 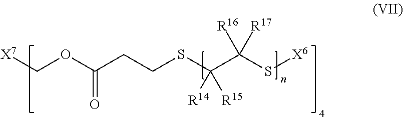
wherein:
R¹⁴, R¹⁵, R¹⁶, and R¹⁷ are independently hydrogen, a branched saturated C₁-C₁₂ hydrocarbyl, (CH₂)_{Y}—H, (CH₂)_{Y}—O—(CH₂)_{Y}′-H, (CH₂)_{Y}—S—(CH₂)_{Y}′-H, CH₂—O—R¹⁸, or CH₂—S—R¹⁸, and wherein R¹⁸ is a functionalized aromatic group or a thiophenyl group;X⁶ is hydrogen, R¹⁸, R¹⁹, (CH₂)_{Y}—H, (CH₂)_{Y}—O—(CH₂)_{Y}′-H, (CH₂)_{Y}—S—(CH₂)_{Y}—H, (CH₂)_{Y}—OH, (CH₂)_{Y}—O—(CH₂)_{Y}′-OH, CH₂CH₂COOR¹⁹, CH₂CH₂CON(H) R¹⁹, wherein R¹⁹ is a branched saturated C₁-C₂₀ hydrocarbyl;
X⁷ is C or Si;
Y and Y¹ range from 1 to 20; and
n is 1 to 100.
